(19) United States
(12) Patent Application Publication
(10) Pub. No.: US 2022/0381789 A1
(43) Pub. Date: Dec. 1, 2022

(54) NANOCOMPOSITE PARTICLES COMPRISING A BORONIC ACID MOIETY AND METHODS FOR PRODUCING AND USING THE SAME

(71) Applicant: Arizona Board of Regents on Behalf of the University of Arizona, a body corporate, Tucson, AZ (US)

(72) Inventors: Craig A. Aspinwall, Tucson, AZ (US); Zeinab Mohktari, Tucson, AZ (US); Jon T. Njardarson, Tucson, AZ (US); Colleen M. Janczak, Tucson, AZ (US); Kevin Scott, Tucson, AZ (US)

(73) Assignee: Arizona Board of Regents on Behalf of the University of Arizona, a body corporate, Tucson, AZ (US)

(21) Appl. No.: 17/315,688

(22) Filed: May 10, 2021

(51) Int. Cl. G01N 33/66

(52) U.S. Cl. CPC G01N 33/66; G01N 2400/00

(57) ABSTRACT

The present invention provides compositions and methods for determining a saccharide level in a sample. In particular, compositions and methods of the invention include a boronic acid moiety that forms a complex with a saccharide.

### CROSS-REFERENCE TO RELATED APPLICATIONS

This application claims the priority benefit of U.S. Provisional Application No. 62/910,428, filed Oct. 3, 2019, which is incorporated herein by reference in its entirety.

### STATEMENT REGARDING FEDERALLY FUNDED RESEARCH

This invention was made with government support under Grant Nos. R21 EB019133 awarded by NIH and 1807343 awarded by NSF. The government has certain rights in the invention.

### FIELD OF THE INVENTION

The present invention relates to compositions and methods for determining a saccharide level in a sample. In particular, compositions and methods include a boronate moiety that forms a complex with a saccharide.

### SUMMARY OF THE INVENTION

Some aspects of the invention are based on determining the level of saccharides in a sample using a boronic acid moiety. In one particular aspect of the invention provides a silica shell encapsulated composite material comprising a boronic acid functional group, which is capable of binding to one or more hydroxyl groups present in the saccharide. One particular composition is illustrated schematically in FIG. 1, where each R is independently H or alkyl, and n is 1 or 2. In some embodiments, n is 1. Yet in another embodiment, n is 2. It should be appreciated that while the composite material illustrated in FIG. 1 shows a plurality of polymers (3) and boronic acid functional groups (6), the scope of the invention is not limited to such a configuration. In fact, the boronic acid functional group (6) can be attached directly to the silica shell (2) and/or each of the linkers (5), which is optionally present, can have one, two, three, or more boronic acid functional groups (6) attached thereto.

Referring again to FIG. 1, the silica shell encapsulated composite material comprises an organic polymer core portion (1); a silica shell portion (2) encapsulating said organic polymer core portion (1); a polymer (3) attached to the surface of said silica shell portion (2); and a boronic acid functional group (6) attached to the surface of said silica shell portion (2). In some embodiments, the boronic acid functional group or a boronate moiety (i.e., B(OR)₂)ₙ) is attached to the surface of the silica shell portion (2) optionally via a linker (5). The linker (5) can be connect to a plurality of boronic acid functional groups (6), typically one or two, often 1 boronic acid functional group (6). In some embodiments, the linker (5) comprises at least two boronic acid functional groups (6), i.e., n=2 or more. In another embodiment, a silane terminated boronic acid is added to the silica shell portion (2). In this manner, the boronic acid functional group (6) becomes an integral part of the silica shell portion (2).

In some embodiments, the silica shell encapsulated composite material is a microcomposite material. As used herein, the term “microcomposite material” refers to composite materials having an average or mean particle size from about 1 μm to about 1,000 μm, typically from about 1 μm to about 500 μm, often from about 1 μm to about 100 μm. In one particular embodiment, the mean particle size of the microcomposite material ranges from about 1 micron to about 10 microns, typically from about 1 micron to about 5 microns, and often about 1 micron to about 3 microns.

Yet in other embodiments, silica shell encapsulated composite material is a nanocomposite material. As used herein, the term “nanocomposite material” refers to a composite material having an average particle size ranging from about 20 nm to about 1500 nm, typically from about 50 nm to about 500 nm, and often from about 100 nm to about 300 nm.

Still in other embodiments, the organic polymer core portion (1) further comprises at least one scintillator material. Such incorporation of scintillator material is disclosed in commonly assigned U.S. Provisional Patent Application No. 62/803,448, filed Feb. 9, 2019 (“the '448 Application”), which is incorporated herein by reference in its entirety. Suitable scintillator materials include, but are not limited to, p-terphenyl (PTP); 1,4-bis (4-methyl-5-phenyl-2-oxazolyl)benzene (dimethyl POPOP); 1 1-phenyl-3-(2,3,6-trimethylphenyl)-2-pyrazoline (PMP); tris(1,3-diphenyl-1,3-propanedionato)-(1,10-phenanthroline) europium (III); diphenylanthracene; 1,4-bis(5-phenyl-2-oxazolyl)benzene; 1,4-bis(2-methylstyryl)benzene; anthracene; and a mixture thereof.

Exemplary polymers (3) that are useful in compositions of the invention include a material selected from the group consisting of polyethylene glycol (PEG), polyethylenimine (PEI), polyvinyl alcohol (PVA), polyvinylcarbazole (PVK), polyethylene oxide, polyacrylamide, agarose, and a combination thereof.

When present, the linker (5) is attached to the silica shell portion (2) via a functional group (4). Exemplary functional groups that can be used to attach the linker to the silica shell portion include, but are not limited to, a hydroxyl group, an amine group, a thiol group, a chloro-silane group, an alkoxy-silane group, a carboxylate group, an ester, an imide group, an isothiocyanate group, and a halide.

Typical organic polymers that are used for the organic polymer core portion (1) include, but are not limited to, polystyrene, polyvinyltoluene (PVT), polyphenylethers (PPE), polyvinyl carbazole (PVK), or a combination or mixture thereof.

The silica shell encapsulated composite materials can be either micro-sized composite materials or nano-sized composite materials. As used herein, the term micro-sized composite material (i.e., microcomposite material) refers to those composite materials of the invention having an average or mean particle size from about 1 μm to about 1,000 μm, typically from about 1 μm to about 500 μm, often from about 1 μm to about 100 μm. In some embodiments, microcomposite material of the invention has an average particle size ranging from about 1 μm to about 10 μm, typically from about 1 μm to about 5 μm, and often from about 2 μm to about 3 μm. The term nano-sized composite material (i.e., nanocomposite material) refers to composite materials of the invention having an average or mean particles size of from about 1 nm to about 1,000 nm, typically from about 1 nm to about 500 nm, often from about 10 nm to about 250 nm.

In some embodiments, the organic polymer core portion (1) has mean particle size of about 300 nm or less. Still in other embodiments, D₉₀ particle size of the organic polymer core portion (1) is about 1000 nm or less.

Yet in other embodiments, the average particle size of said organic polymer core portion (1) ranges from about 20 nm to about 1500 nm. Still in other embodiments, the thickness of said silica shell portion (2) ranges from about 10 nm to about 250 nm.

Typical thickness of silica shell portion (2) is no more than about 10 nm, often no more than about 50 nm, more often no more than about 100 nm, still more often no more than about 200 nm, and most often no more than about 500 nm.

As used herein, the term “about” is not intended to limit the scope of the invention but instead encompass the specified material, parameter or step as well as those that do not materially affect the basic and novel characteristics of the invention. When referring to a numerical value, the terms “about” and “approximately” are used interchangeably herein and refer to being within an acceptable error range for the particular value as determined by one of ordinary skill in the art, which will depend in part on how the value is measured or determined, e.g., the limitations of the measurement system, i.e., the degree of precision required for a particular purpose. For example, the term “about” typically means within 1 standard deviation, per the practice in the art. Alternatively, the term “about” can mean ±20%, typically ±10%, often ±5% and more often ±1% of the numerical value. In general, however, where particular values are described in the application and claims, unless otherwise stated, the term “about” means within an acceptable error range for the particular value.

### BRIEF DESCRIPTION OF THE DRAWINGS

FIG. 1 is a schematic illustration of one embodiment of a nanocomposite material of the invention.

FIG. 2A is a picture of Alizarin Red S displacement assay for determination of D-glucose concentration showing change in color of Alizarin Red S from red (λₘₐₓ=517 nm) to orange (λₘₐₓ=475 nm) by binding to boronic acid and from orange to red through displacement from the boronic acid complex by D-glucose.

FIG. 2B is UV-VIS spectra of free (solid red line) and boronic acid-bound (dashed orange line) Alizarin Red S.

FIG. 2C is fluorescence emission spectra of free (red diamonds) and boronic acid-bound (orange circles) Alizarin Red S (λₑₓ₌470 nm).

FIG. 3 is a graph showing binding of CBBA to saccharides obtained by Alizarin red S displacement assay. The decrease in fluorescence emission of ARS-CBBA complex (λₑₘ=585 nm, λₑₓ=470 nm) was observed with the addition of monosaccharides: maltose (red squares), glucose (green circles), mannose (blue triangles), galactose (pink diamonds), and fructose (purple x's). The highest affinity of CBBA was observed with fructose. Data are normalized to the fluorescence of ARS-CBBA complex.

FIG. 4 shows scintillation response of PS-APTS10%-CBBA nanoSPA (red circles) to ³H-D-glucose. A Glucose-Mix solution was prepared by combining ³H-D-glucose with D-glucose (³H-D-glucose=2.5×10⁻⁴%). Scintillation response increased with increasing concentration of ³H-D-glucose. The NPE observed with control samples (PS-APTS10% nanoSPA, blue squares) is due to free ³H-D-glucose molecules which increased with increasing concentration of ³H-D-glucose.

FIG. 5 shows scintillation response of PS-APTS25%-CBBA nanoSPA (red circles) to ³H-D-glucose. A Glucose-Mix solution was prepared by combining ³H-D-glucose with D-glucose (³H-D-glucose=3.6×10⁻⁵%) and titration was performed at high mM concentrations of D-glucose. Scintillation response increased with increasing concentration of ³H-D-glucose. The NPE observed with control samples (PS-APTS25% nanoSPA, blue squares) is due to free ³H-D-glucose molecules which increased with increasing concentration of ³H-D-glucose.

FIG. 6 shows scintillation response of PS-APTS25%-CBBA nanoSPA (red circles) to ³H-D-glucose. A Glucose-Mix solution was prepared by combining ³H-D-glucose with D-glucose (³H-D-glucose=6.3×10⁻⁴%) and titration was performed at low mM concentration of D-glucose. Scintillation response increased with increasing concentration of ³H-D-glucose. The NPE observed with control samples (PS-APTS25% nanoSPA, blue squares) is due to free ³H-D-glucose molecules which increased with increasing concentration of ³H-D-glucose.

FIG. 7 shows scintillation response of PS-APTS50%-CBBA nanoSPA (red circles) to ³H-D-glucose. A Glucose-Mix solution was prepared by combining ³H-D-glucose with D-glucose (³H-D-glucose=2.5×10⁻⁴%). Scintillation response increased with increasing concentration of ³H-D-glucose. The NPE observed with control samples (PS-APTS50% nanoSPA, blue squares) is due to free ³H-D-glucose molecules which increased with increasing concentration of ³H-D-glucose.

FIG. 8 is a TEM image of scintillant PS-APTS core-shell microparticles.

FIG. 9 shows scintillation response of PS-APTS10%-CBBA microparticles (red circles) to ³H-D-glucose. A Glucose-Mix solution was prepared by combining ³H-D-glucose with D-glucose (³H-D-glucose=2.5×10⁻⁴%). Scintillation response increased with increasing concentration of ³H-D-glucose. The NPE observed with control samples (PS-APTS10% microparticles, blue squares) is due to free ³H-D-glucose molecules which increased with increasing concentration of ³H-D-glucose.

FIG. 10 shows scintillation response of PS-MPTS-VPBA nanoSPA (red circles) to ³H-D-glucose. D-glucose solution was prepared by combining ³H-D-glucose with D-glucose (³H-D-glucose=2.0×10⁻⁴%). Scintillation response increased with increasing concentration of ³H-D-glucose. The NPE observed with control samples (PS-MPTS nanoSPA, blue squares) is due to free ³H-D-glucose molecules which increased with increasing concentration of ³H-D-glucose.

FIG. 11 shows scintillation response of PS-MPTS-APBA nanoSPA (red circles) to ³H-D-glucose. PS-MPTS-APBA nanoparticles were prepared using a Mal-PEG₂-NHS crosslinker to immobilize APBA moieties on the surface of nanoparticles. A Glucose-Mix solution was prepared by combining ³H-D-glucose with D-glucose (³H-D-glucose=1.1×10⁻⁴%). Scintillation response increased with increasing concentration of ³H-D-glucose. The NPE observed with control nanoSPA (PS-MPTS nanoSPA, blue squares) is due to free ³H-D-glucose molecules which increased with increasing concentration of ³H-D-glucose.

FIG. 12 shows scintillation response of 3-aminophenyl boronic acid-functionalized nanoparticles (PS-TEPI-APBA nanoparticles, red circles) to ³H-D-glucose. A Glucose-Mix solution was prepared by combining ³H-D-glucose with D-glucose (³H-D-glucose=2×10⁻⁴%). Scintillation response increased with increasing concentration of ³H-D-glucose. The NPE observed with control samples (PS-TEPI nanoparticles, blue squares) is due to free ³H-D-glucose molecules which increased with increasing concentration of ³H-D-glucose.

FIG. 13A is UV absorption spectra of AFBA in PBS buffer (pH 7.4) with increasing concentration of D-glucose. (B) The absorbance of AFBA at 245 nm decreased through binding to D-glucose.

FIG. 13B is absorption spectra of free AFBA at 245 nm showing the absorption decreased through binding of AFBA to D-glucose (K_{d}˜100 mM).

FIG. 14A is UV absorption spectra of AFBA in PBS buffer (pH 7.4) with increasing concentration of D-fructose.

FIG. 14B is absorption spectra of free AFBA at 245 nm showing the absorption decreased through binding to D-fructose (K_{d}˜2 mM).

FIG. 15A is UV absorption spectra of diBA4b in PBS buffer (pH 7.4) with increasing concentration of D-glucose.

FIG. 15B is absorption spectra of free diBA4b at 245 nm showing the absorption decreased through binding to D-glucose (K_{d}˜6 mM).

FIG. 16A is UV absorption spectra of diBA4b in PBS buffer (pH 7.4) with increasing concentration of D-fructose.

FIG. 16B is absorption spectra of free diBA4b at 245 nm showing the absorption decreased through binding to D-fructose (K_{d}˜2 mM).

FIG. 17 is a schematic illustration of a structure of PS-MPTS-diBA4b nanoSPA and the scintillation response of diBA4b-nanoSPA (red circles) to ³H-D-glucose. D-glucose solution was prepared by combining ³H-D-glucose with D-glucose (³H-D-glucose=1.25×10⁻⁴%). Scintillation response increased with increasing concentration of ³H-D-glucose. PS-MPTS nanoSPA (blue squares) was used as a control. The NPE, observed with control samples, is due to free ³H-D-glucose molecules and increased with increasing concentration of ³H-D-glucose.

FIG. 18 shows an electron microscopy of diBA4b-nanoSPA. Image labelled (A) is a STEM image of PS-MPTS-diBA4b nanoparticles. STEM-EDX images of PS-MPTS-diBA4b nanoparticles for elemental analysis of C, O, and Si are labelled as (B), (C), and (D), respectively.

FIG. 19 is a schematic illustration of a structure of DiBA-nanoSPA and the scintillation response of diBA-nanoSPA (red circles) to ³H-D-glucose. D-glucose solution was prepared by combining ³H-D-glucose with D-glucose (³H-D-glucose=1.25×10⁻⁴%). Scintillation response increased with increasing concentration of ³H-D-glucose. PS-APTS nanoSPA (blue squares) was used as a control. The NPE, observed with control samples, is due to free ³H-D-glucose molecules and increased with increasing concentration of ³H-D-glucose.

FIG. 20 is a schematic illustration of a structure of DiBA-nanoSPA (PS-TEOS-diBA4bSi nanoSPA) and the scintillation response of diBA-nanoSPA (red circles) to ³H-D-glucose. D-glucose solution was prepared by combining ³H-D-glucose with D-glucose (³H-D-glucose=2×10⁻⁴%). Scintillation response increased with increasing concentration of ³H-D-glucose. PS-APTS nanoSPA (blue squares) was used as a control. The NPE, observed with control samples, is due to free ³H-D-glucose molecules and increased with increasing concentration of ³H-D-glucose.

### DETAILED DESCRIPTION OF THE INVENTION

Saccharides are important biomolecules that are involved in numerous physiological and pathological processes in our cells. These small carbohydrate molecules are building blocks of more complex molecules, such as DNA, RNA, and ATP, and provide energy for many biological processes. D-glucose is a monosaccharide that is required for cells to function. However, the uncontrolled concentration of blood D-glucose leads to complications and disorders such as diabetes, which in turn causes more severe health problems such as heart attack, limb amputation, and blindness. This chronic metabolic disorder motivates the development of more robust and stable sensors for in vitro and in vivo analysis of D-glucose.

As used herein, the terms “saccharide,” “sugar,” and “carbohydrate” are used interchangeably herein and generally refers to a mono- and disaccharide. A carbohydrate is a biomolecule consisting of carbon (C), hydrogen (H) and oxygen (O) atoms, usually with a hydrogen—oxygen atom ratio of 2:1 (as in water) and thus with the empirical formula Cm(H₂O)n (where m may be different from n). The carbohydrates can exist as an aldose and/or a ketose form. The term “monosaccharide” refers to any type of hexose of the formula C₆H₁₂O₆ or a derivative thereof. The ring structure (i.e., ring type) of the monosaccharide can be a pyranose or a furanose. In addition, the monosaccharides can be an α- or β-anomer. Monosaccharide can be a ketonic monosaccharide (i.e., ketose), an aldehyde monosaccharide (i.e., aldose), or any type of hexose of the formula C₆H₁₂O₆ or a derivative thereof. Exemplary aldoses include, but are not limited to, allose, altrose, glucose, mannose, gulose, idose, galactose, talose, ribose, arabinose, xylose, lyxose, and derivatives thereof. Exemplary ketoses include, but are not limited to, psicose, fructose, sorbose, tagatose, ribulose, xylulose, and derivatives thereof. The term “disaccharide” refers to a carbohydrate composed of two monosaccharides. It is formed when two monosaccharides are covalently linked to form a dimer. The linkage can be a (1→4) bond, a (1→6) bond, a (1→2) bond, etc. between the two monosaccharides. In addition, each of the monosaccharides can be independently an α- or β-anomer. Exemplary disaccharides include, but are not limited to, sucrose, lactose, maltose, trehalose, cellobiose, lactulose, chitobiose, etc. Each of the monosaccharides can independently be a ketonic monosaccharide (i.e., ketose), an aldehyde monosaccharide (i.e., aldose), or any type of hexose of the formula C₆H₁₂O₆ or a derivative thereof. Other examples of disaccharides include, but are not limited to, allose, altrose, glucose, mannose, gulose, idose, galactose, talose, ribose, arabinose, xylose, lyxose, psicose, fructose, sorbose, tagatose, ribulose, xylulose, etc. Each monosaccharide can also be independently an (L)-isomer or a (D)-isomer. As a specific example, lactose is a disaccharide found in animal milk. It consists of a molecule of D-galactose and a molecule of D-glucose bonded by beta-1-4 glycosidic linkage.

Another aspect of the invention provides a method for determining the level of a saccharide present in a sample. The method includes: contacting the sample with a silica shell encapsulated composite material disclosed herein in the presence of a signal generator under conditions sufficient to produce a signal when the saccharide is present in the sample; and analyzing the signal to determine the level of saccharide present in the sample. The signal generator can be a separate component or it can be incorporated into the silica shell encapsulated composite material, such as in proximity assay, including scintillation proximity assay.

Samples that can be analyzed using the compositions and methods of the invention include any biological samples such as, but not limited to, blood, serum, saliva, urine, and live cells, cell lysates, microorgans, or tissue.

As will be expected, the type of signal analzyed depends on the signal generator that is used. Exemplary signals that can be used include, but are not limited to, fluorescence, phosphorescence, UV-Vis absorption, infrared absorption, Raman spectroscopy, scintillation, radioisotope decay and electrochemistry. Exemplary signal generting materials includes, but are not limited to, alizarin red S (ARS), NBD-glucose (2-(N-(7-nitrobenz-2-oxa-1,3-diazol-4-yl) amino)-2-deoxyglucose), radiolabeled glucose, and 2-methylaniline.

In one particular embodiment, the signal generator produces radioisotope decay signal.

As discussed herein, the signal generator can be incorporated within the organic polymer core portion. Such incorporation of the signal generator, for example, using a scintillator material, allows detection or determination of a saccharide level via a proximity assay.

In one particular embodiment, methods of the invention provide determining the level of D-glucose from a sample. Despite the significance of the detection of D-glucose and D-glucose-containing biomolecules, such as glycated proteins, robust sensors are not available for D-glucose analysis. Enzymatic sensors, based on the enzymatic oxidation of D-glucose and production of electrochemically active species, have been developed for blood D-glucose analysis. Enzymatic assays for D-glucose analysis rely on the catalytic activity of one or more enzymes, which makes them expensive and unstable.

Compositions and the methods of the invention allow a significantly more sensitive, and/or cost effective non-enzymatic detection or determination of D-glucose level in samples of interest. Accordingly, methods of the invention are more accurate and more suitable for analytical applications. While there are other conventional non-enzymatic D-glucose sensors, typically these sensors require receptor molecules designed for selective and sensitive detection of D-glucose.

In particular, some aspects of the invention provide compositions and methods for determining the level of saccharide present in a sample using a boronic acid moiety, i.e., B(OR)₂, where R is H or alkyl, typically C₁-C₈ alkyl. Boronic acids are widely applied in chemistry and biochemistry, including but not limited to protection, enrichment of biomolecules, inhibition of enzymes, materials science and self-assembly, transmembrane transportation, drug delivery, derivatization, bioconjugation, and labeling, immobilization, separation and purification of diols, and recognition of diols, including but not limited to D-glucose and glycated proteins.

Boronic acids are Lewis acids with an electron-accepting center, the vacant orbital of boron, and form a complex with Lewis bases with electron-donating groups, such as hydroxyl groups. The complexation of neutral boronic acids with hydroxyl groups leads to the production of boronate anions as illustrated in Scheme 1 below. The geometry of boron changes from trigonal planar to tetrahedral in this acid-base reaction. pKₐ of boronic acids change upon binding to diols, and the Lewis acid becomes more acidic due to the electron deficiency of boron in the boronic acid complex with diols (i.e., boronate anions).

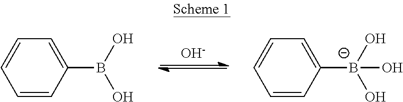

Boronic acids and their boronate anions reversibly form 5- or 6-membered ring esters with cis-1,2- and trans-1,3-diols through covalent bonds. See, for example, Scheme 2 below. The ester products of boronic acids are hydrolytically less stable than those of the boronate anions so that the binding of boronic acids to diols occurs at a pH slightly above the pKₐ of the boronic acid. Therefore, any modification in the structure of the boronic acid which leads to lower pKₐ improves the stability of the ester complex. Reversible covalent binding of boronic acids to cis-diols enables dynamic monitoring of important biological processes, such as D-glucose metabolic pathway and glycosylation of proteins. Without being bound by any theory, it is believed that the complexed saccharide in the ester form is in equilibrium with its free form and any fluctuation in the concentration of free saccharides translates into the extent of complexation with boronic acids.

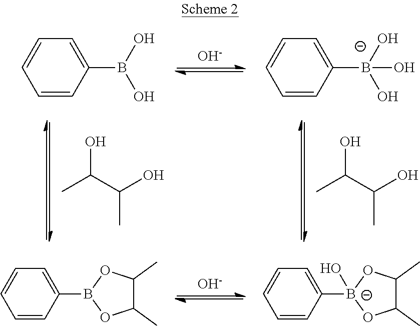

Some aspects of the invention are based on the discovery of processes by the present inventors that the level of saccharides and/or glycoproteins can be monitored using boronic acid-based sensors. In particular using a silica shell encapsulated composite material disclosed herein.

Boronic acids are commonly used as recognition moieties in saccharide-responsive sensors in a variety of platforms including but not limited to hydrogels, copolymers, polyelectrolytes, nanoparticles, electrochemical sensors, and molecularly imprinted polymers. Many monoboronic acid (monoBA) sensors were synthesized for homogeneous or heterogeneous analysis of D-glucose by optical, electrochemical, electrochemiluminescence, or vibrational techniques.

In general, the stability of esters formed by covalent binding of boronic acids to cis-1,2-diols is greater than that of trans-1,3-diols, which determines the order of affinities of saccharides to boronic acids. Usually, fructose has the highest binding affinity to boronic acid moieties, compared to D-glucose and other monosaccharides. The higher affinity of boronic acid to fructose is not a problem in biological samples since the concentration of D-glucose 10-1000 times higher than those of other monosaccharides, including fructose. Again without being bound by any theory, due to steric constraints boronic acids do not bind to trans-1,2-diols of saccharides. D-glucose binds to boronic acids through its cis-1,2- and trans-4,6-diols to form S-and 6-membered ester rings. Scheme 3 below shows the binding modes of D-glucose with boronic acid.

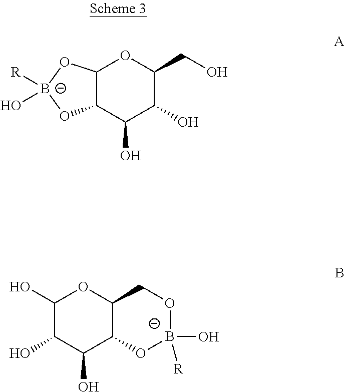

Diboronic acids (diBA) have higher affinities to D-glucose, compared to monoBAs, because diBAs can bind to two pairs of cis-diols in D-glucose. K_{d} values for monoBA and diBA are in the order of 100 mM and 10 mM, respectively. DiBA is a better binding moiety for D-glucose analysis at low mM concentrations, which makes it more suitable for D-glucose analysis in biological samples such as cell lysates and live cells, where few mM concentration of D-glucose is observed. In a 1:1 bisdentate complex of a diBA with D-glucose, the saccharide molecule is bound to both boronic acid moieties. By increasing concentration of D-glucose, a 1:2 complex is formed with cis-1,2-diols bound to boronic acid moieties, as illustrated in Scheme 4.

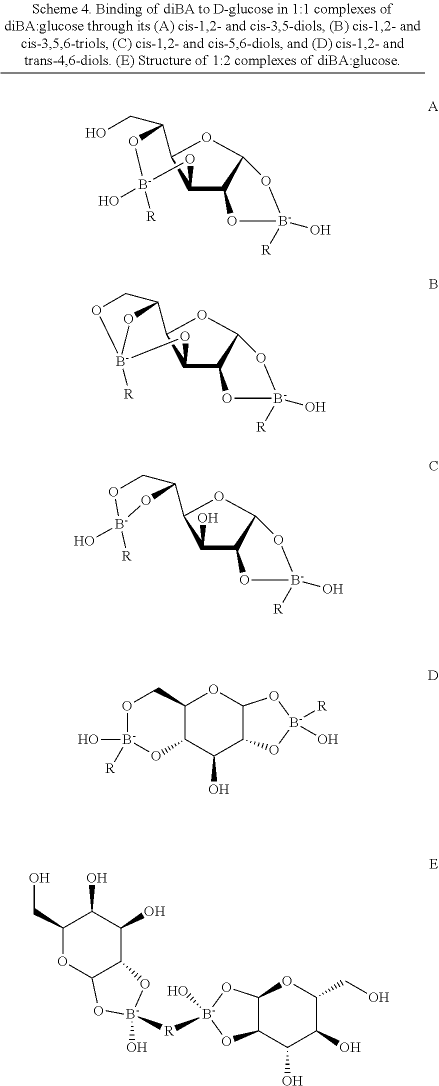

Without being bound by any theory, it is believed 1-OH and 6-OH are essential in the binding of D-glucose to boronic acid moieties in aqueous media. As revealed by nuclear magnetic resonance (NMR) analysis, diBAs may bind to D-glucose through all five hydroxy groups, depending on time and environment. It has been observed by the present inventors that D-glucose binds to monoBAs through 1,2-diols and 3,5,6-triols, and to diBAs through 1,2- and 5,6-diols, in aqueous media.

Commercially available diBAs possess two boronic acid groups that are helpful in diol capping to protect the diol groups. However, these diBAs do not facilitate binding to two pairs of cis-diols in a single D-glucose molecule due to the lack of specific orientation of the monoBA moieties that facilitate a suitable spatial disposition to hydroxyl groups, necessitating synthesis of glucose-sensitive diBAs.

Some aspects of the invention provide nanoparticle scintillation proximity assay (“nanoSPA”) sensors for detecting and/or determining the level of saccharide (e.g., D-glucose) present in a sample. The nanoSPA of the invention utilizes boronic acids as a saccharide recognition moiety. In one particular embodiment, nanoSPA functionalized with monoBAs and diBAs (monoBA-nanoSPA and diBA-nanoSPA) are utilized for analysis of D-glucose at low mM concentration, by mixing unlabeled and tritiated D-glucose (D-glucose-[6-³H(N)]). ³H-D-glucose has the same structure and physicochemical properties as regular D-glucose. Binding of D-glucose to monoBAs and diBAs is a reversible and dynamic process, which makes nanoSPA of the invention suitable for time-resolved monitoring of D-glucose in live cells.

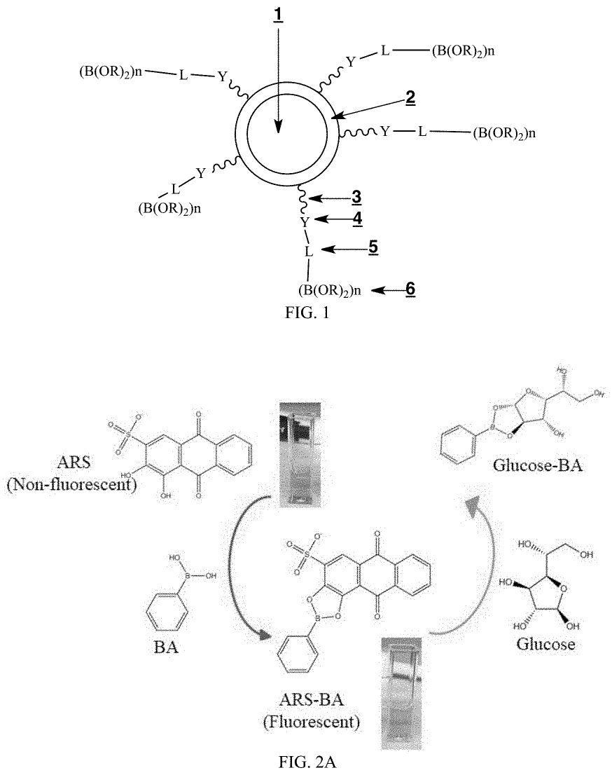

β-particle emitting atoms, such as ³H, ¹⁴C, ³²P, ³³P, and ³⁵S, are important molecular labels due to their small size and the prevalence of these atoms in biomolecules but are challenging to selectively detect and quantify within aqueous biological samples and systems. Another aspect of the invention provides a silica shell encapsulated composite material-based scintillation proximity assay platform (nanoSPA) for the separation-free, selective detection or determination of radiolabeled saccharides. As disclosed above, saccharides can be labelled with tritium (³H). In one particular example, nanoSPA particles were prepared by incorporating scintillant fluorophores into polystyrene core particles and encapsulating the scintillant-doped cores within functionalized silica shells. Such nanoSPA are also functionalized with boronic acids on its surface as illustrated in FIG. 1. The functionalized silica shell surface (e.g., with a functional group “Y” in FIG. 1) enabled covalent attachment of boronic acid moieties (optionally via a linker “L”). Such nanoSPA boronic acid materials can be used to detect ³H-labeled saccharides such as D-glucose as well as other saccharides. In this manner, 1 nmol quantities of D-glucose can be detected directly in a sample.

In some embodiments, a hydrophilic polymer (polymer (3) in FIG. 1) is present on the surface of silica shell encapsulated composite material. Without being bound by any theory, it is believed that the presence of this hydrophilic polymer on the silica shell encapsulated composite material's surface significantly reduces non-specific binding or hydrophobic interaction with hydrophobic materials that may be present in the sample, thereby resulting in a significant increase in accuracy and reliability of saccharide detection using silica shell encapsulated composite materials of the invention. It is believed that using a hydrophilic polymer can reduce non-specific binding by at least about 25%, typically by at least about 50%, and often at least about 80%.

General procedure for producing silica shell encapsulated composite materials can be found in commonly assigned U.S. patent application Ser. No. 15/798183, filed Oct. 30, 2017 (“the '183 Application”). For attaching a polymer to the surface of the silica shell encapsulated composite material, more specifically to the outer surface of the silica shell portion, a wide variety of methods can be used. For example, after the silica shell portion is made, the functional group that is present on the surface of the silica shell portion can be used to attach the polymer (3) and/or the linker (5) and/or a silane containing the boronic acid groups (6). Alternatively, a first silica shell layer is made. This first silica shell layer encapsulates the organic polymer core portion (1). To this first silica shell layer, another layer of silica shell can be added that incorporates the polymer (3) and/or a silane containing the boronic acid groups (6). For example, using a mixture of tetraethylorthosilicate (TEOS) and silane containing polymer (e.g., polyethylene glycol-triethoxysilane (PEG-Op13 Si(OEt)₃), and/or silane containing the boronic acid groups (e.g., silane functionalized boronic acid, compound 1 in the Examples section below)) one can form a second or the outer layer of the silica shell. In this manner, the polymer becomes an integral part of or embedded within the silica shell structure.

In some embodiments, the silica shell encapsulated composite material includes a surface functional group (4), i.e., “Y” in FIG. 1, on the silica shell portion. This allows attachment of a wide variety of different boronic acid moieties optionally via a linker. This functionalization allows one to tailor the silica shell encapsulated composite material to attach a wide variety of boronic acid moieties. Generally, a hydrophilic polymer, in particular organic hydrophilic polymer, is used in the composition of the invention. Hydrophilic polymers are well known to one skilled in the art. In general, hydrophilic polymers are polymers in which at least a portion of the polymer dissolves in water. Exemplary polymers that are useful in compositions of the invention include, but are not limited to, polyethylene glycol (PEG), polyethylenimine (PEI), polyacrylamide, agarose, polyvinyl alcohol, polyethylene oxide, as well as a combination thereof, and other hydrophilic organic polymers known to one skilled in the art.

In some embodiments, at least about 2%, typically at least about 25%, often at least about 50%, and more often at least about 90% of the surface area of silica shell nanocomposite material comprises the polymer (3). Alternatively, at least about 20%, typically at least about 50%, and often at least about 95% of the functional group present on the surface of the silica shell encapsulated composite material is attached to the polymer (3).

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

Styrene, alumina, p-terphenyl (pTP), and 1,4-bis(4-methyl-5-phenyl-2-oxazolyl)-benzene (dimethyl POPOP) were purchased from Acros Organics (NJ). Tetraethylorthosilicate (TEOS), (3-mercaptopropyl)trimethoxysilane (MPTS), (3-aminopropyl)triethoxysilane (APTES), polyvinylpyrrolidone (PVP, 40,000 average molecular weight), 2,2′-azobis(2-methylpropionamidine) dihydrochloride (AIBA), N-(3-dimethylaminopropyl)-N′-ethylcarbo-diimide hydrochloride (EDAC HCl), N-hydroxysuccinimide (NHS), biotin-PEG-NHS (Mw=3000) and Tween-20 were obtained from Sigma Aldrich (St. Louis, Mo.). Dimethylsulfoxide was purchased from Fisher Chemical (Hampton, N.H.). PEG₆₋₉-dimethylchlorosilane (90%) was purchased from Gelest (Morrisville, Pa.). Isopropyl alcohol (IPA), chloroform, aqueous ammonium hydroxide (28%) and acetonitrile were obtained from EMD Millipore (Billerica, Mass.). Ethyl alcohol was purchased from Decon Laboratories (King of Prussia, Pa.). BioCount liquid scintillation cocktail was purchased from Research Products International (Mt. Prospect, Ill.). ³H biotin (25.0 Ci/mmol) and ³H sodium acetate (1.59 Ci/mmol) was purchased from Perkin Elmer (Waltham, Mass.). Streptavidin was obtained from VWR (Radnor, Pa.). NeutrAvidin was purchased from Pierce Thermo Scientific (Waltham, Mass.), and Nanosep centrifugal devices with 3 kDa MWCO membranes were purchased from Sartorius (Gottingen, Germany). All chemicals except styrene were used as received. Inhibitor was removed from styrene by passing the monomer through a 0.5 cm diameter by 3 cm long alumina column immediately prior to use.

All scintillation measurements were performed using a Beckman LS 6000 IC scintillation counter.

Precautions: All radioactive materials should be handled and disposed according to federal, state, and local guidelines. Styrene is flammable, is an irritant, and is a health hazard. Ethyl acetate, ethyl alcohol, isopropyl alcohol, TEOS and AIBA and acetonitrile are flammable and are irritants. MTPS and pTP are irritants and hazardous to aquatic life. APTES is corrosive and an irritant. EDAC HCl and PEG₆₋₉-dimethylchlorosilane are irritants. Chloroform exhibits acute toxicity and is a health hazard. Ammonium hydroxide solution is corrosive, an irritant, and hazardous to aquatic life.

General Synthesis of boronic acid compounds: A representative example of preparation of boronic acid compounds are shown in the scheme below.

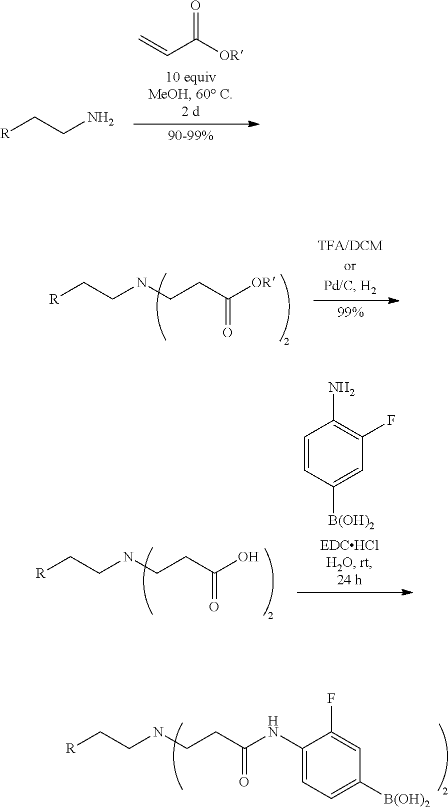

In this manner, some of the representative boronic acid compounds that can be prepared or were prepared include, but are not limited to, the following compounds:

| | R = | R′ = |
|---|---|---|
| 1 | 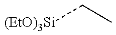 | Bn |
| 2 | 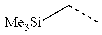 | Bn |
| 3 | 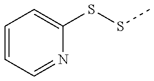 | tBu |
| 4 | 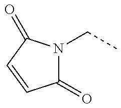 | tBu |

### Exemplary Synthesis of Boronic Acid Compounds

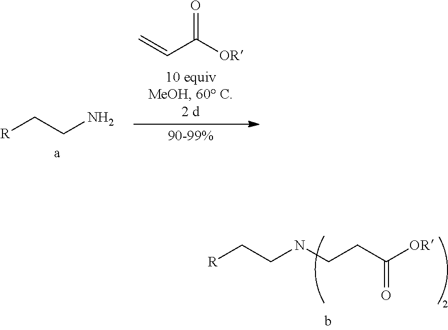

Amines of the form a were either synthesized according to published literature or purchased from commercial vendors. Such amines were added slowly to a solution of acrylic esters (either benzyl acrylate or tent-butyl acrylate) in freshly distilled methanol at 0° C. The reaction mixture was warmed to room temperature and stirred in the dark for 48 hours. After reaction was determined to be complete, the solvent was removed by rotary evaporation, and the crude reaction mixture was purified by silica gel chromatography to obtain the desired product. In the case, when the R group contained a silane moiety, the R group was removed and the product either crystallized or precipitated for purification.

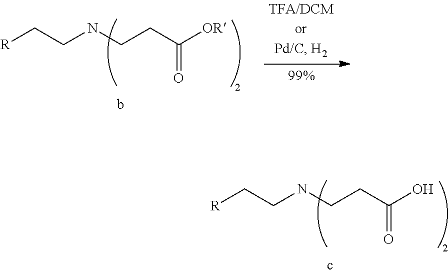

Substituted amines of the form c were generated by deprotection of the analogous amines of form b. In the case that R′ was tent-butyl, the starting material was taken into a 2:5 mixture of trifluoroacetic acid:dichloromethane (“TFA:DCM”) to a final concentration of 0.1 M. After completion of the reaction as determined by thin layer chromatography (“TLC”), the solvents were removed under reduced pressure. Ice cold ether was added to precipitate the product, and this solid was further washed to remove any additional impurities. In the case that R′ was benzyl, the starting material was taken into a 1:1 mixture of ethyl acetate and methanol to a final concentration of 0.25 M, and 10% w/w palladium on carbon was added to the mixture. Hydrogen gas was applied at 1000 psi using a Parr hydrogenator. Upon completion of the reaction, the mixture was filtered through charcoal and celite and washed once with methanol, after which the solvents were removed under reduced pressure to give the pure product.

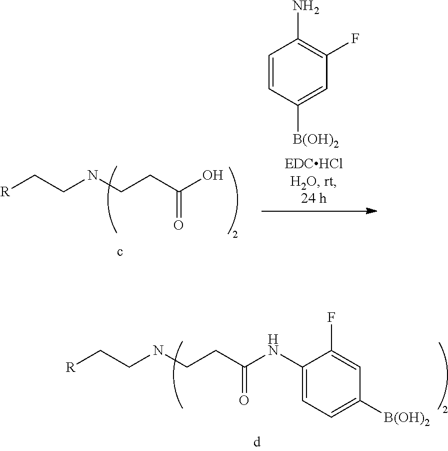

Compounds of the form c were combined with 3 equivalents of ethylenediamine carbodiimide, hydrochloride salt (“EDC⋅HCl”), and 3 equivalents of 4-amino-3-fluorophenylboronic acid, and water to a final concentration of 0.25 M of diacid. After stirring for 4 hours an additional equivalent of 4-amino-3-fluorophenylboronic acid was added. The mixture was allowed to stir overnight, at which point solvent was removed under reduced pressure to give the crude product.

Synthesis of silane functionalized diboronic acid. Silane functionalized diboronic acids were synthesized as described above and shown schematically below:

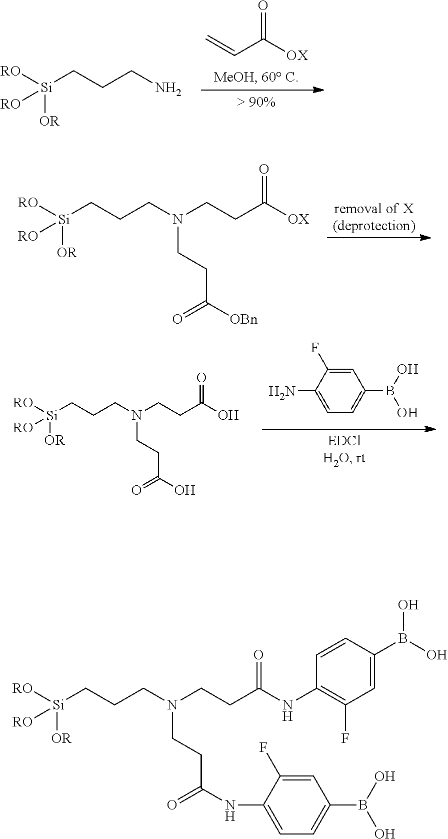

Where R and X represent methyl, ethyl, propyl, t-butyl, phenyl, benzyl (—CH₂Phenyl), allyl, —CH₂SiMe₃, —CH₂-aryl, —CH₂CCl₃, alkyl, aryl, heteroaryl functionalities. Compound 1 was specifically synthesized as:

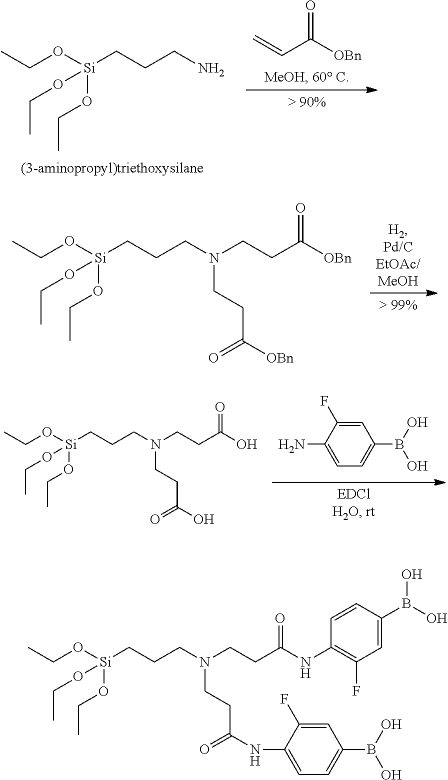 [SMILES: CCO[Si](CCCN)(OCC)OCC ; C=CC(=O)OCc1ccccc1 ; CCO[Si](CCCN(CCC(=O)OCc1ccccc1)CCC(=O)OCc1ccccc1)(OCC)OCC ; CCO[Si](CCCN(CCC(=O)O)CCC(=O)O)(OCC)OCC ; Nc1ccc(B(O)O)cc1F ; CCO[Si](CCCN(CCC(=O)Nc1ccc(B(O)O)cc1F)CCC(=O)Nc1ccc(B(O)O)cc1F)(OCC)OCC]

Preparation of silica shell encapsulated composite material particles: Scintillant fluorophore-doped PS core particles were prepared following the protocol described previously. Briefly, styrene (3 g, inhibitor-free) was added to 100 mL degassed H₂O in an Ar-flushed 500 mL round-bottomed flask heated to 70° C. in an oil bath. Polymerization was initiated by adding 10 mg of AIBA dissolved in approximately 200 μL of H₂O to the reaction flask. The H₂O/styrene mixture was stirred rapidly for at least 6 h. Excess styrene and some H₂O were removed from the nanoparticle solution under reduced pressure using a rotary evaporator to reduce the total volume to approximately 100 mL. A 1-2 mL aliquot of the solution was removed and lyophilized to determine the mass-based concentration (mg/ml) of polystyrene core nanoparticles.

PS particles were doped with scintillant fluorophores by dissolving 53 mg (135 mmol) of dimethyl POPOP and 262 mg (1.14 mmol) of pTP in 20 mL of 1:9 isopropyl alcohol/chloroform (v:v). Scintillant fluorophores dissolved in solvent were added directly to the aqueous PS particle solution in a 500 mL round-bottomed flask. The particle solution was agitated using a bath sonicator for several minutes to disperse organic solvent droplets throughout the H₂O and the solution was stirred rapidly for at least 1 h. Organic solvents were then removed under reduced pressure using a rotary evaporator.

Silica shells were added to scintillant fluorophore-doped PS particles by dispersing 2 mL PS particle stock solution (approximately 56 mg nanoparticles) in 200 mL isopropyl alcohol with 38 mL H₂O. For hydroxyl- or thiol-functionalized silica shells, 7.5 mL NH₄OH was added to the flask. For amine functionalized shells, 5.0 mL NH₄OH was added to the flask. The dispersion was stirred briskly for several minutes while either 2.0 mL of TEOS or a mixture containing 1.8 mL TEOS and 0.2 ml of either APTES or MPTS was added dropwise. Stirring was continued for 1 h before particles were collected by centrifugation and rinsed several times with H₂O.

Preparation of Large Polystyrene Particles: Large (5-30 μm) polystyrene (PS) microparticles were prepared by using PVP as a stabilizer during the polymerization process. Styrene (5 g, inhibitor-free) and PVP (2.5 g) were dissolved in a mixture of 5 mL of water and 35 mL of ethyl alcohol in an Ar-flushed 500 mL round-bottomed flask heated to 70° C. in an oil bath. Polymerization was initiated by adding MBA (1 g) to the reaction flask. The mixture was stirred rapidly for 6 h, after which 75 mL of water was added to the flask. Excess styrene and some H₂O were removed from the nanoparticle solution under reduced pressure using a rotary evaporator to a final volume of approximately 50 mL. Large particles were separated from nanoparticles by centrifugation at 16000×g for 10 min, followed by removal of supernatant, and re-dispersion of the pelleted particles in 50 mL water. The cycle of centrifugation and re-dispersion was repeated a total of 5 times. The microparticles were finally dispersed in 25 mL of water, and a 1 mL aliquot was removed and lyophilized to determine the weight per volume. The particles were later doped with scintillant fluorophores and coated with silica shells following the procedures outlined in the article.

Saccharide Detection: D-glucose-[6-³(N)] was purchased from Perkin Elmer (Waltham, Mass.) with a specific activity of 45.7 Ci/mmol and concentration of 1.0 mCi/mL. Tetraethoxysilane (TEOS, 98%), 3-aminopropyl triethoxysilane (APTS, 99%), 3-mercaptopropyl trimethoxysilane MPTS (97%), 3-(triethoxysilyl) propyl isocyanate (TEPI), 2,2′-azobis-2-methyl-propanimidamide, dihydrochloride (MBA, 97%), sodium hydrogen phosphate (99%), D-glucose, N-(3-dimethylaminopropyl)-N′-ethylcarbodiimide hydrochloride (EDC), N-Hydroxysuccinimide (NHS), 4-vinylphenylboronic acid (VPBA), 3-amino phenylboronic acid (APBA), 4-amino-3-fluorophenyl boronic acid (AFBA), and alizarin red S (ARS) were purchased from Sigma Aldrich (St. Louis, Mo.). Maleimide-PEG₂-succinimidyl ester was purchased from Quanta Biodesign Ltd. (Plain City, Ohio). Styrene (99%), polyvinylpyrrolidone (PVP), para-terphenyl (pTP, 99%), and 4-bis(4-methyl-5-phenyl-2oxyzolyl) benzene (dmPOPOP, 98%), chloroform, and alumina were purchased from ACROS Organics (Geel, Belgium). ACS-grade isopropyl alcohol (IPA) and tetrahydrofuran (THF) were purchased from Merck (Kenilworth, N.J.). BioCount liquid scintillation cocktail was purchased from Research Products International (Mt. Prospect, Ill.). ACS-grade ammonium hydroxide was purchased from EMD Millipore (Billerica, Mass.). Sodium hydroxide (98.9%) was purchased from Avantor Performance Materials (Center Valley, Pa.). 4-(2-carboxyethyl) benzene boronic acid (CBBA, 97%) was purchased from Alfa Aesar. DiBA4b was synthesized as described elsewhere. Purified H₂O (18 MΩcm) was obtained using Easy Pure UV/UF (Barnstead).

Fabrication and Characterization of monoBA-nanoSPA and diBA-nanoSPA for Glucose Analysis: nanoSPA was prepared and characterized, as described above. PS-TEOS, PS-APTS, and PS-MPTS nanoparticles were utilized for the development of D-glucose sensors based on BA-decorated nanoSPA platforms. Modifications of nanoSPA using commercial monoBAs and synthetic diBAs were performed to evaluate nanoSPA for sensitive and selective detection of D-glucose.

### Coupling of monoBAs and diBAs to nanoSPA

Immobilization of CBBA on NH₂-nanoSPA: 7.3 mg CBBA was dissolved in 1 mL solution of 1:1 EtOH:MES (20 mM, pH 5). A mixture of 3 mg EDC and 3.7 mg NHS dissolved in 1 mL MES was added to the solution of CBBA with shaking for 2 h. The mixture of CBBA, EDC, and NHS was added to a suspension of NH₂-nanoSPA (PS-APTS10%, 25%, or 50%) in 13 mL PBS buffer (100 mM, pH 7.4) at room temperature overnight. CBBA-nanoSPA (PS-APTS-CBBA) was collected by one cycle of centrifugation and rinsing with EtOH and 3 cycles of rinsing with H₂O, followed by resuspension in PBS.

Immobilization of VPBA on PS-MPTS Nanoparticles: A solution of 9.5 mg VPBA in 500 μL EtOH was mixed with a suspension of thiol-functionalized (PS-MPTS) nanoparticles in PBS for 2 h with shaking. VPBA-functionalized nanoparticles were collected by one cycle of centrifugation and rinsing with EtOH and 3 cycles of rinsing with H₂O. Nanoparticles were resuspended in PBS.

Immobilization of APBA on PS-MPTS Nanoparticles: A solution of 8.7 mg APBA in 500 μL EtOH was mixed with 17 mg Mal-PEG₂-NHS in 2 mL citrate buffer (20 mM, pH 5) and incubated at room temperature overnight. A suspension of thiol-functionalized (PS-MPTS) nanoparticles in 15 mL PBS buffer (pH 7.4) was mixed with the crosslinker-conjugated APBA with shaking for 1 h. APBA-decorated nanoparticles were collected by 1 cycle of centrifugation and rinsing with EtOH and 3 cycles of rinsing with H₂O, followed by resuspension in PBS.

Immobilization of APBA on PS-TEOS Nanoparticles: 15 mg APBA and 200 TEPI were dissolved in 1 mL THF and incubated overnight at room temperature with shaking. 30 mg PS nanoparticles were coated using APBA-conjugated TEPI and 1.8 mL TEOS. PS-TEPI-APBA nanoparticles were collected by 1 cycle of centrifugation and rinsing with EtOH and 3 cycles of rinsing with H₂O, followed by resuspension in PBS buffer.

Immobilization of diBA4b on PS-MPTS Nanoparticles: diBA4b (about 20 mg) with a free thiol group was dissolved in 14 mL mixture of 30:70 EtOH:PBS. 2 mL of the resultant solution was incubated with a suspension of thiol-functionalized (PS-MPTS) nanoparticles in PBS for 2 h. DiBA4b-decorated nanoparticles were collected by 1 cycle of centrifugation and rinsing with EtOH and 3 cycles of rinsing with H₂O, followed by resuspension in PBS buffer.

Immobilization of diBA4b on PS-APTS Nanoparticles: A suspension of NH₂-nanoSPA in citrate buffer was incubated with 22 mg Mal-PEG₂-NHS crosslinker overnight at room temperature with shaking. Crosslinker-decorated nanoparticles were collected with one cycle of centrifugation and resuspension in PBS buffer. 2 mL solution of 20 mg diBA4b in 14 mL mixture of 30:70 EtOH:PBS was mixed with the crosslinker-decorated nanoparticles for 1 h. DiBA4b-decorated nanoparticles were collected by 1 cycle of centrifugation and rinsing with EtOH and 3 cycles of rinsing with H₂O, followed by resuspension in PBS buffer.

Immobilization of DiBA4bSi on PS-TEOS Nanoparticles: diBA4b Si (about 33 mg) with a silane functional group was dissolved in 10 mL IPA and used in the silica coating step in addition to 1.8 mL TEOS to coat 30 mg PS core nanoparticles. DiBA4bSi-decorated nanoparticles were collected by 1 cycle of centrifugation and rinsing with EtOH and 3 cycles of rinsing with H₂O, followed by resuspension in PBS buffer.

Alternatively, DiBA4bSi or other silane-functionalized boronic acids can be immobilized on PS-TEOS nanoparticles by first preparing core-shell nanoparticles as described in [0052]. Core-shell nanoparticles (100 mg) were rinsed three times in dry acetonitrile and suspended in 5 mL dry acetonitrile with 2% (w/v) silane-functionalized boronic acid. Suspended particles were mixed for at least 12 hours before being rinsed three times with acetonitrile, 3 times with ethanol, and three times with water before being finally suspended in water or buffer.

Characterization of CBBA Binding to Saccharides Using ARS Displacement Assay: 200 μL solution of 9.6 mM CBBA in EtOH was mixed with 1500 μL solution of 100 ARS in PBS buffer. The resultant ARS-BA complex was titrated using 50 and 500 mM solutions of 5 saccharides, D-glucose, D-fructose, D-galactose, D-maltose, and D-mannose, up to 270 mM total concentration of the saccharides. Fluorescence emission spectra of the ARS-BA complex were collected for each addition of the saccharide solution from 530 to 620 nm by excitation at 470 nm. Fluorescence emission at 585 nm was measured as a function of the concentration of saccharide.

Characterization of Binding of AFBA and diBA4b to Monosaccharides Using UV-VIS Spectroscopy of the Boronic Acids: 2 mL solution of 230 μM AFBA in PBS or 65 diBA4b in 30:70 EtOH:PBS was titrated with a solution of 1 M D-glucose and 1 M D-fructose up to total assay concentration of 600 or 60 mM monosaccharide, respectively. UV absorbance spectra of the AFBA and diBA4b were collected from 215 to 305 nm and the change in maxima at 245 nm was followed as a function of the concentration of monosaccharides.

Detection of ³H-D-glucose Using monoBA- and diBA-nanoSPA: A Glucose-Mix was prepared by mixing a solution of D-glucose with ³H-D-glucose to raise the concentration of D-glucose in the SPA experiments. Three replicates of a 1-mL suspension of nanoparticles in PBS buffer (1 mg core/mL) were used for all samples for detection of ³H-D-glucose. monoBA-or diBA-functionalized nanoparticles were titrated with aliquots of the Glucose-Mix and scintillation response of the nanoparticles was measured as a function of the total concentration of D-glucose (D-glucose and ³H-D-glucose) and the activity of ³H-D-glucose. Total assay concentrations of D-glucose and activities of ³H-D-glucose in each experiment are represented in titration plots.

Scanning Transmission Electron Microscopy of diBA4b-nanoSPA: Elemental analysis of the surface of diBA4b-functionalized nanoparticles was performed using an Ultra-High Resolution 1-30 kV scanning transmission electron microscope SU9000 with energy dispersive X-ray (EDX) detector (Hitachi, Ltd., Tokyo, Japan) at 30 kV accelerating voltage. STEM grid samples were prepared by deposition of 10 μL aliquot of a dilute dispersion of nanoparticles in H₂O on copper grids coated with carbon films (Structure Probe Inc., West Chester, Pa.). The excess suspension was wiped off after 10 min and the grids were dried in a desiccator for at least 2 h before electron microscopy.

X-Ray Photoelectron Spectroscopy of diBA4b-nanoSPA: Elemental analysis of the surface of diBA4b-functionalized nanoparticles was performed using a Kratos Axis 165 Ultra X-ray Photoelectron Spectrometer/Surface Analysis system under a pressure less than 1×10⁻⁸ Torr. A monochromatized Al Kα source was used with incident energy of 1486.6 eV. The system was operated at 15 keV and 20 mA emission current. Lyophilized (powdered) nanoparticles were loaded onto stainless steel sample holders using double-sided carbon sticky tape. Low energy electron beam was used for charge compensation, to remove any excess charge retention on nanoparticles. All low-resolution survey data were collected with a pass energy of 160 eV over a binding energy range of 0 eV to 1200 eV with 50 eV increments. Individual regions were monitored using high resolution scanning at a pass energy of 20 eV and 1 eV increments over a binding energy range of 160-696 eV. More specifically, F1s, C1s, N1 s, S2p, and B1s were scanned over the binding energy ranges of 680-696, 280-300, 392-408, 160-172, and 184-200 eV, respectively. Photoelectrons were amplified and detected using a microchannel plate stack in a Z-configuration with position sensitive detection using delay-line detector.

Sample Preparation: All SPA samples were prepared in PBS buffer (100 mM, pH 7.4). All concentrations are reported based on the total assay volume (1 mL) including the nanoparticles. A Glucose-Mix was prepared using D-glucose and ³H-D-glucose for titrating the nanoSPA platforms with monoBA or diBA-functionalized surface. nanoSPA concentration in all samples was 1 mg PS core/mL. To measure the concentration of PS core nanoparticles a 1-mL suspension of PS core in H₂O was freeze-dried and the mass of resultant powder was measured. A minimum of three sample replicates was prepared in all experiments.

Data Collection, Analysis, and Presentation: A Beckman Coulter LS 60001C liquid scintillation analyzer was used for scintillation response measurements. Scintillation responses are collected for a minute per sample and reported as emitted photon counts per minute (CPM). All results are presented as mean±standard deviation of data collected from at least three sample replicates. Error bars in all plots are representative of the standard deviation. A Tecnai G2 Spirit 20-120 kV transmission electron microscope was used for imaging the microparticles. An Ultra-High Resolution 1-30 kV scanning transmission electron microscope SU9000 with EDX detector (Hitachi, Ltd., Tokyo, Japan) was used for imaging and elemental analysis of boronic acid-functionalized nanoparticles at 30 kV accelerating voltage.

Results and Discussion: D-glucose sensors were developed based on homogeneous scintillation proximity assays. nanoSPA was modified with boronic acid moieties, for selective and sensitive detection of ³H-D-glucose. Commercial monoBA and synthetic diBA compounds were utilized on the surface of nanoSPA. Scheme 5 illustrates the structure of CBBA, VPBA, and APBA utilized for fabrication of monoBA-nanoSPA, and AFBA that was used for the synthesis of diBA4b and diBA4bSi, used in the architecture of diBA-nanoSPA.

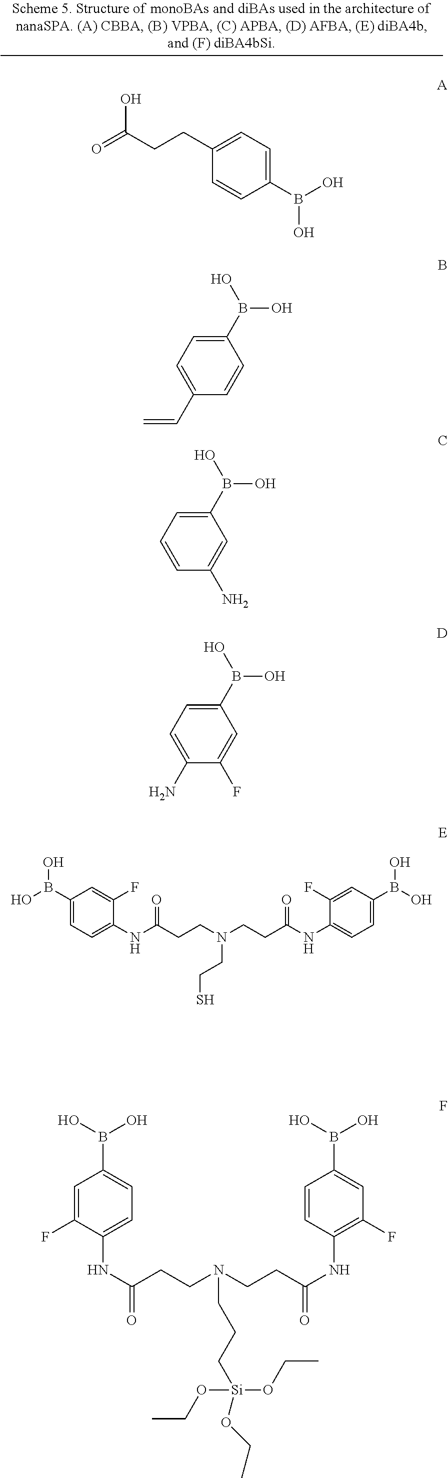

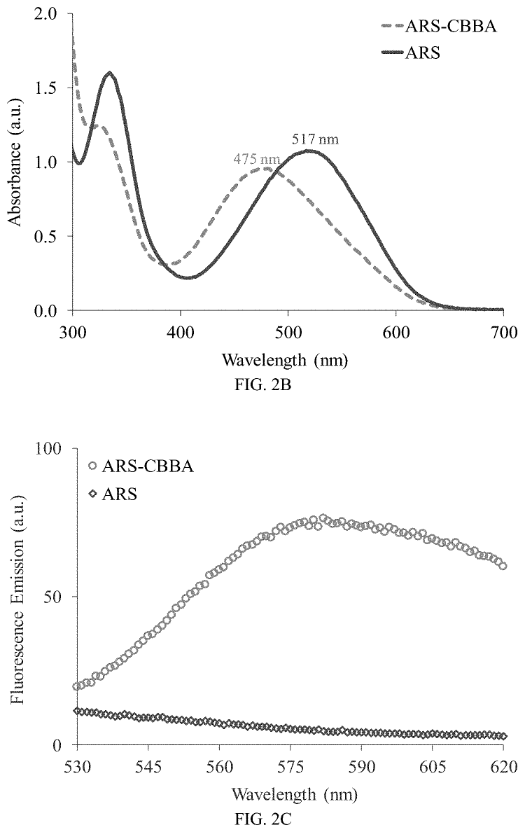

Evaluation of the Binding of CBBA to Saccharides Using ARS Displacement Assay by Fluorimetry: ARS is a commonly used fluorogenic dye in the label-free detection of saccharides using boronic acids. ARS is a red non-fluorescent dye at physiological pH and forms an orange fluorescent complex through binding to boronic acids (ARS-BA). Titration of the complex ARS-BA with other diols (e.g. D-glucose) displaces the ARS and leads to restoration of the original color of non-fluorescent ARS. FIG. 2A illustrates a schematic representation of the ARS displacement assay.

UV-VIS spectroscopy or fluorimetry are both utilized to follow the change in optical properties of ARS-BA complex as a function of the concentration of D-glucose. FIG. 2B shows the UV-VIS spectrum of ARS-BA complex with maximum absorbance at 517 nm which shifts to 475 nm upon displacement of ARS. Fluorescence emission of ARS-BA complex with maximum emission at 585 nm quenches upon complete displacement of ARS by D-glucose, FIG. 2C.

ARS displacement assay was employed to investigate the affinity of a monoBA (CBBA) to selected saccharides, before immobilizing it as the binding moiety on nanoSPA. Increasing concentrations of D-glucose, D-fructose, D-galactose, D-maltose, and D-mannose were added to a solution of CBBA and ARS and the fluorescence emission of the ARS-CBBA complex was monitored at 585 nm as a function of the total assay concentrations of the saccharides. The intensity of fluorescence emission of ARS-CBBA complex decreased and approached zero by the addition of saccharides.

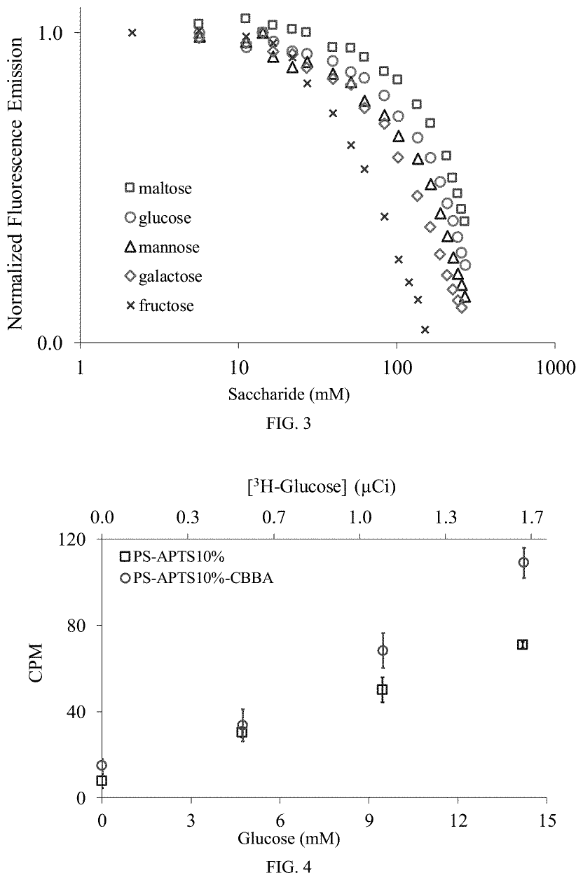

FIG. 3 illustrates the normalized fluorescence emission of ARS-CBBA as a function of the concentration of saccharides. This plot shows that the fluorescence of ARS-CBBA complex decreases with different rates for the selected saccharides which suggests that CBBA has different affinities to the experimented saccharides with the highest affinity to fructose. CBBA has a much lower affinity to D-glucose, as expected for a monoBA. Nonetheless, CBBA was utilized to fabricate a sensor for ³H-D-glucose by immobilizing it on amine-functionalized (PS-APTS) nanoparticles through peptide coupling.

Application of monoBA-nanoSPA for Detection of ³H-D-glucose: CBBA has a carboxyl group on the phenyl ring and was immobilized on NH₂-nanoSPA through peptide coupling by first activating the carboxyl groups of CBBA and then coupling carboxyl-activated CBBA to the amine groups of nanoSPA. CBBA-functionalized nanoSPA (PS-APTS-CBBA) was used for the detection of ³H-D-glucose. Federal regulations limit the amount of ³H-D-glucose to nmoles in research labs. On the other hand, monoBAs have low affinity to D-glucose, with K_{d} values in the high mM range. Therefore, a Glucose-Mix was used to raise the concentration of D-glucose to mM range and facilitate measurement of the specific binding of monoBA-functionalized nanoparticles to this monosaccharide. The Glucose-Mix was used to titrate the CBBA-functionalized nanoSPA up to assay concentration of 14 mM and activity of 1.6 μuCi. The scintillation responses of the PS-APTS-CBBA nanoSPA was measured as a function of the total concentration of D-glucose, including both regular and radiolabeled D-glucose, and activity of ³H-D-glucose. A control group was prepared using PS-APTS nanoSPA without the D-glucose binding moiety.

FIG. 4 illustrates the results of the titration of monoBA-nanoSPA and control nanoSPA using a Glucose-Mix with 2.5×10⁻⁴mole percentage of ³H-D-glucose. The scintillation response of PS-APTS-CBBA nanoSPA increased with increasing concentration of D-glucose due to binding to ³H-D-glucose in addition to the NPE due to unbound ³H-D-glucose molecules that reside close to the scintillant nanoparticles in the solution. The NPE on control nanoparticles also increased with the concentration of D-glucose because of more activity of ³H-D-glucose added to the mixture. Signal enhancement on PS-APTS-CBBA, compared to PS-APTS nanoSPA, was approximately 1.5. Poor signal/background ratio observed using this sensor platform is due to the usage of Glucose-Mix. There is a much higher probability of binding regular D-glucose, compared to the radiolabeled D-glucose, due to the extremely low mole percent of ³H-D-glucose in the Glucose-Mix. Additionally, the low affinity of CBBA to D-glucose limits the detection of ³H-D-glucose and there are unbound ³H-D-glucose molecules in the mixture. Higher concentrations of the Glucose-Mix are needed to improve the extent of binding to PS-APTS-CBBA nanoSPA. However, the limited concentration of ³H-D-glucose makes the first problem more pronounced. Furthermore, the surface coverage of the nanoparticles with the monoBA may be improved to increase the sensitivity of the nanoparticles to D-glucose.

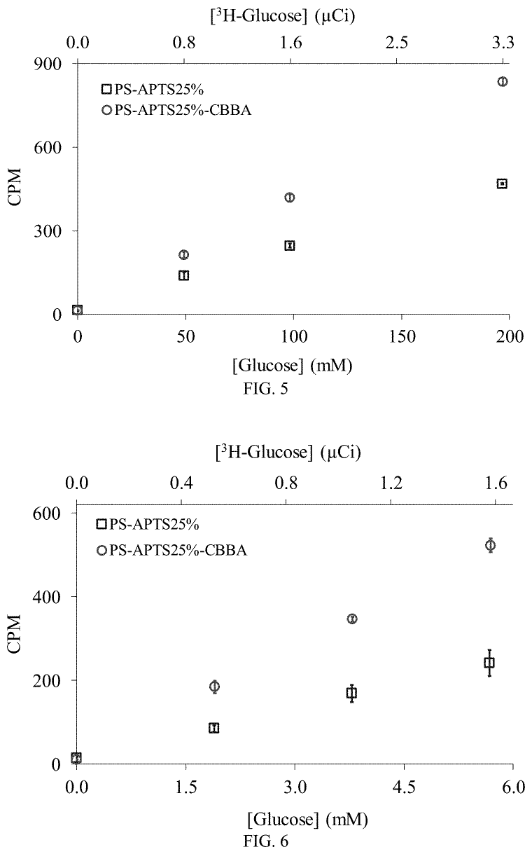

To improve the sensitivity of PS-APTS-CBBA nanoSPA to D-glucose, a higher concentration of APTS was used to coat the PS core and immobilize a greater number of CBBA molecules on the surface of core-shell nanoparticles. Usually, 10% APTS and 90% TEOS is used in the process of silica coating. 25% APTS and 75% TEOS was utilized while fixing all other conditions to the previous experiment. PS-APTS25% nanoSPA was functionalized with the same monoBA to obtain PS-APTS25%-CBBA nanoSPA platform. Titration of PS-APTS25%-CBBA and control nanoSPA without the monoBA functionality (PS-APTS25%) with a Glucose-Mix was performed by adding aliquots of the Glucose-Mix to the suspension of nanoparticles. Aliquots of the Glucose-Mix was added to the suspension of nanoparticles up to 200 mM (3.2 μCi). Scintillation responses of the nanoparticles are shown in FIG. 5.

Similar trends were observed with PS-APTS25%-CBBA nanoSPA as the experiment performed using PS-APTS10%-CBBA nanoSPA. The scintillation responses of both groups of nanoparticles increased but the high NPE, observed with the control group, demonstrated the same issues observed in the previous experiment. Nonetheless, the signal to background improved to about 1.8 due to the higher concentration of monoBA on the surface of nanoparticles, despite a lower mole percentage of ³H-D-glucose in the Glucose-Mix (3.6×10⁻⁵).

Since low mM concentrations of D-glucose are observed in cells and the D-glucose sensors based on nanoSPA are being developed for potentially intracellular dynamic measurement of this monosaccharide, titration of PS-APTS25%-CBBA nanoSPA was repeated with a Glucose-Mix at low mM concentrations with a higher activity of ³H-D-glucose (6.3×10⁻⁴% ³H-D-glucose). Titration of the PS-APTS25%-CBBA nanoSPA and a control group (PS-APTS25%) was performed up to 6 mM D-glucose (1.6 μCi). The signal to background ratio was improved to 2.2 (FIG. 6).

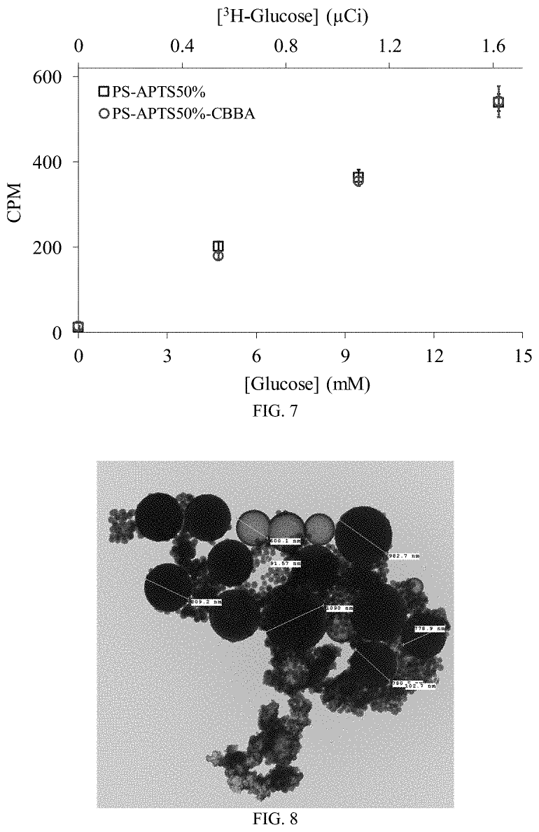

Based on the improvement of signal to background ratio by immobilizing a greater concentration of CBBA on the surface of nanoparticles, using higher concentration of APTS in the silica coating step of the nanoparticles, PS nanoparticles were coated with 50% APTS and 50% TEOS to facilitate immobilization of a greater number of CBBA molecules on the surface of scintillant nanoparticles. PS-APTS50% nanoSPA was functionalized with CBBA and titrated with a Glucose-Mix, up to 15 mM (1.6 μCi). A control group of PS-APTS50% nanoSPA was included in the titration experiment. Signal to background ratio was about 1 (FIG. 7).

To further improve the surface coverage of scintillant particles with CBBA a slightly different platform of D-glucose sensor was developed using scintillant core-shell microparticles instead of nanoparticles. It was believed that higher surface on individual particles would facilitate the immobilization of a greater number of monoBAs and detection of ³H-D-glucose with a greater sensitivity. PS microparticles were fabricated using the procedure described for fabrication of PS nanoparticles, with the following modifications: a mixture of 10 g styrene and 5 g PVP was used in place of 6 g styrene and the polymerization was performed in a mixture of 100 mL EtOH and 10 mL degassed H₂O using 1 g AIBA. The obtained PS microparticles were doped with pTP and dmPOPOP followed by coating with 10% APTS and 90% TEOS. FIG. 8 shows a TEM image of the scintillant PS-core silica-shell microparticles.

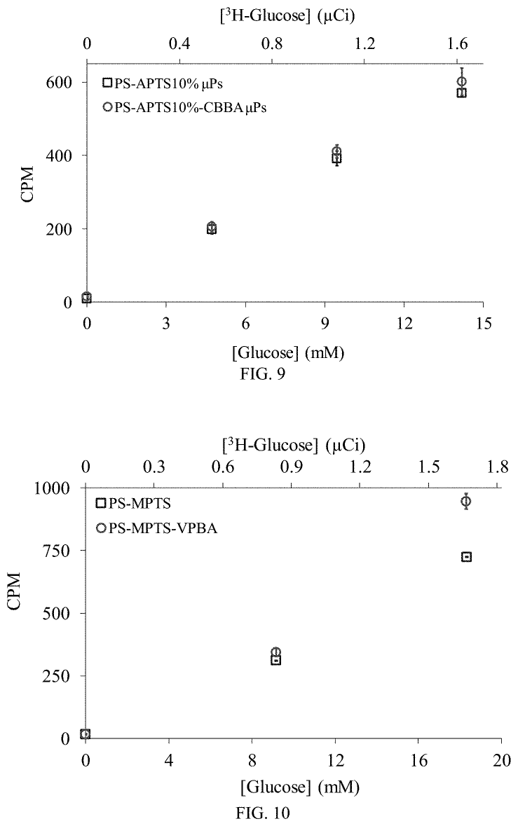

Scintillant core-shell microparticles were functionalized with CBBA and titrated with a Glucose-Mix up to 15 mM (1.6 μCi). A control group of PS-APTS microparticles with no immobilized CBBA was also titrated with the Glucose-Mix. Scintillation responses of the CBBA-functionalized microparticles (PS-APTS 10%-CBBA μPs) were not significantly different from that of the control microparticles (PS-APTS 10% μPs), FIG. 9. The poor signal to background ratio (˜1) may be due to unsuccessful immobilization of CBBA on the surface of microparticles.

Additionally, commercially available monoBAs were explored to improve the sensitivity of monoBA-nanoSPA. VPBA with an alkene group on the phenyl ring was coupled to thiol-functionalized nanoparticles through thiol-ene click chemistry. PS-MPTS-VPBA nanoparticles were tested for D-glucose detection by titration with a Glucose-Mix up to 18 mM (1.7 μCi). A control group of PS-MPTS nanoparticles was included in the SPA experiment. A significant difference of scintillation response was observed between the two groups of nanoparticles, however, a poor signal to background ratio was obtained, approximately 1.3 (FIG. 10). the poor signal enhancement is due to the low affinity of monoBA to D-glucose and extremely low concentration of ³H-D-glucose in the mixture.

To further explore monoBAs on the surface of nanoparticles, a nanoSPA platform was prepared using APBA on PS-MPTS nanoparticles using a heterogeneous crosslinker. This monoBA has an amine group on the phenyl ring and was coupled to thiol-functionalized (PS-MPTS) nanoparticles through the heterobifunctional crosslinker. Mal-PEG₂-NHS ester was used for thiol-ene coupling of the maleimide to thiols and the carboxyl to the amine groups of APBA.

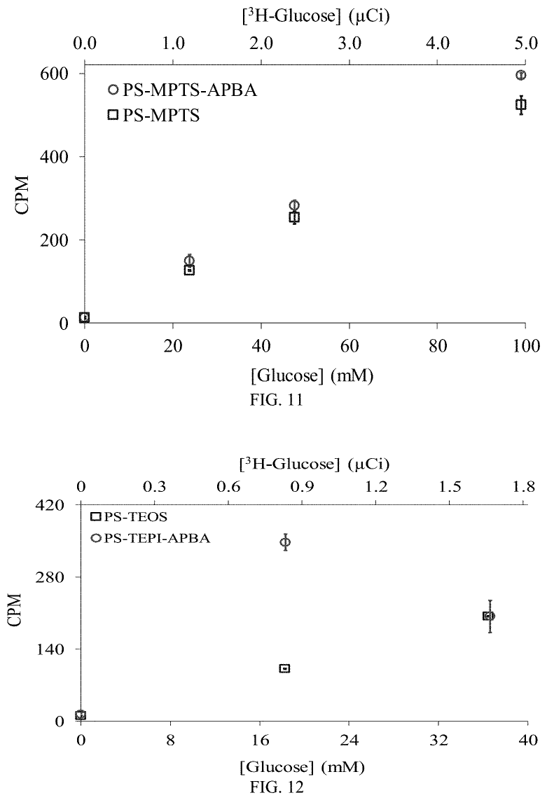

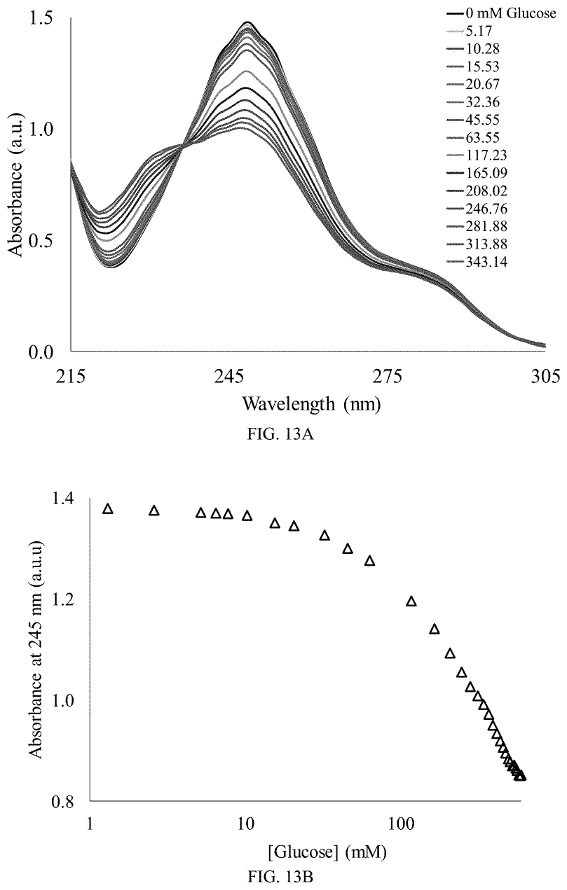

PS-MPTS-APBA nanoparticles were utilized for the detection of ³H-D-glucose by titration with a Glucose-Mix up to 100 mM (5 μCi). A control group of nanoparticles was included in the SPA experiment. A significant difference of scintillation response was observed between the two groups of nanoparticles (FIG. 11), however, a signal to background ratio of approximately 1.1 was obtained, presumably due to the low affinity of APBA to D-glucose and low mole percentage of ³H-D-glucose in the Glucose-Mix.

A different coupling chemistry of APBA to scintillant nanoparticles was utilized based on a fructose molecular imprinting sensor developed using APBA and TEPI as shown below. Mesoporous silica beads were fabricated by fructose-bound APBA molecules conjugated to TEPI. The fructose molecular imprinting sensor was linearly responsive to fructose, using ARS displacement assay, up to 100 mM.

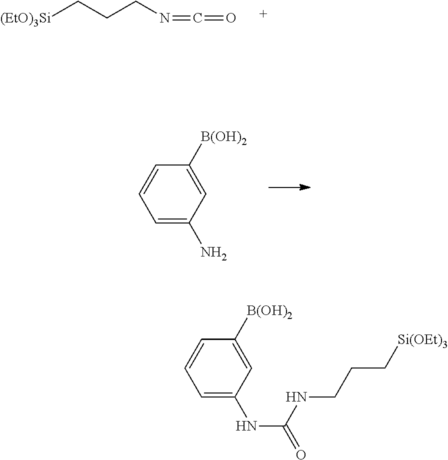

PS-TEPI-APBA nanoparticles were fabricated by coating PS nanoparticles with 10% APBA-conjugated TEPI and 90% TEOS. PS-TEPI-APBA nanoparticles were tested for D-glucose detection by titration with a Glucose-Mix up to 38 mM (1.7 μCi). A control group of PS-TEOS nanoparticles was included in the SPA experiment. There was no significant difference between the scintillation responses of the two groups of nanoparticles and the signal to background ratio was 1 (FIG. 12).

Evaluation of the Binding of AFBA and diBA4b to Saccharides Using the Optical Properties of the BAs: To increase the signal to noise ratio, different platforms of nanoSPA were developed and investigated using higher affinity binding moieties to D-glucose. A diBA-functionalized nanoSPA platform was designed and developed for detection of D-glucose. AFBA was used to synthesize a series of diBA molecules with slightly different structure and K_{d} values to D-glucose. The diBA used for fabrication of diBA-nanoSPA was based on the compound 4b. Boron in boronic acid shares bonds with two hydroxyl groups and an alkyl or aryl group. Usually, the aryl group is modified to incorporate more electron-withdrawing groups, e.g., halogens, to increase the acidity of the boronic acid. Typically, pKₐ ranging from 4.0 to 10.5 is observed for boronic acids, depending on the structure of the alkyl/aryl group attached to boron.

The affinity of diBA4b to D-glucose was studied in solution while conjugation to the surface of the scintillant nanoparticles is currently being optimized. The binding affinity of diBA4b and its precursor monoBA (i.e. AFBA) to D-glucose was evaluated in solution by following the UV absorbance of the boronic acids in PBS buffer at 245 nm as a function of D-glucose concentration. Titration of AFBA and diBA4b with fructose was performed as a reference to validate the tighter binding of D-glucose to diBA4b, compared to AFBA, while the same affinity of AFBA and diBA4b to fructose was expected.

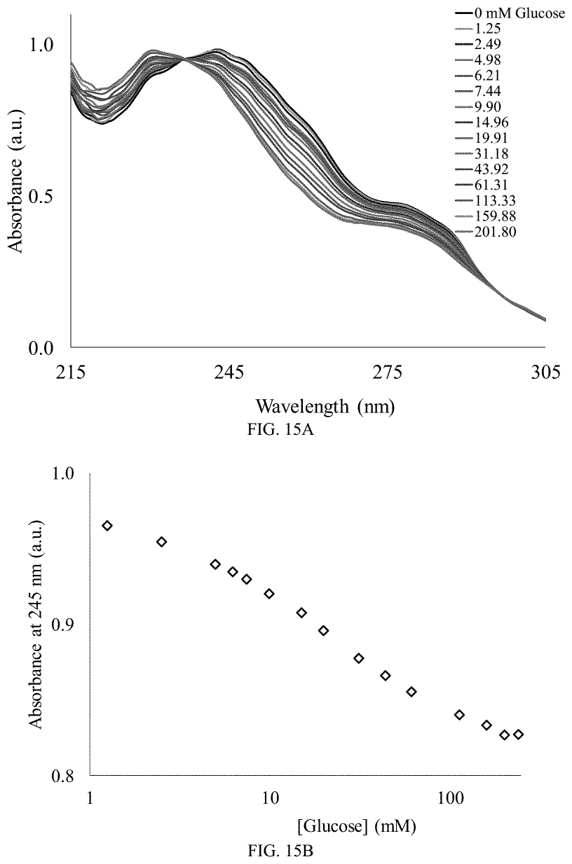

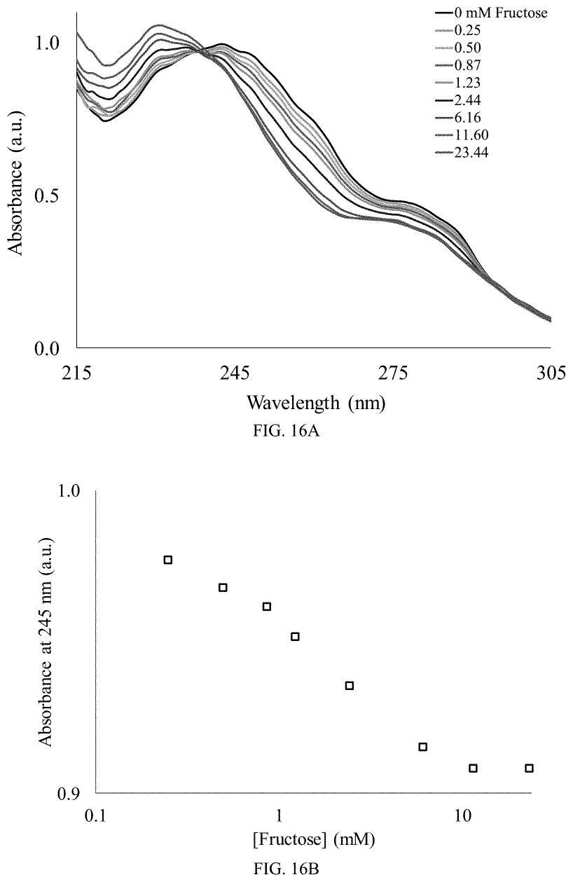

FIG. 15A depicts the change in UV spectra of AFBA upon increasing concentration of D-glucose. Maximum absorbance at 250 nm decreases and the absorbance at 225 nm increases upon the D-glucose addition. The maximum absorbance wavelength of diBA4b shifted from 250 to 225 nm. The absorbance of AFBA at 245 nm was followed as a function of the concentration of D-glucose, as illustrated in FIG. 15B. AFBA has a low affinity to D-glucose with a K_{d} value of 100 mM. The UV absorbance of AFBA at 245 nm decreases upon increasing concentration of D-glucose above 100 mM. These results show that AFBA has a low affinity to D-glucose and would not be a good choice for detection of D-glucose in physiological concentrations. AFBA, however, may be used to assemble diBA4b with tighter binding to D-glucose.

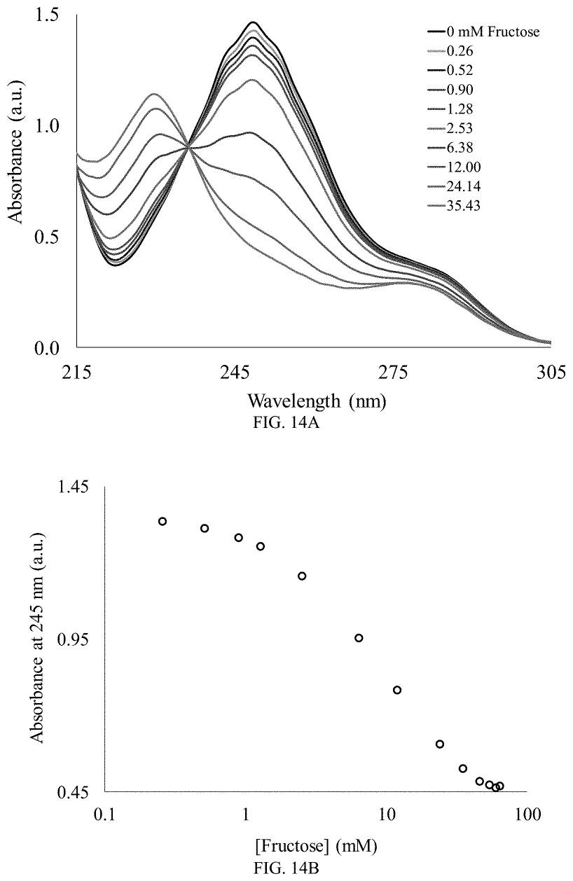

Conversely, monoBAs have higher affinity to fructose, compared to D-glucose, and they are ideally better choices for development of fructose sensors. Titration of AFBA with fructose confirmed the higher affinity. FIGS. 14A and 14B show the change in UV spectra of AFBA upon increasing concentration of fructose and its absorbance at 245 nm as a function of fructose concentration. AFBA has a lower K_{d} for fructose, with the absorbance at 245 nm decreasing dramatically upon increasing fructose concentration.

Titration diBA4b with D-glucose and fructose was done to compare the affinity of AFBA. The reported K_{d} value of approximately 6 mM for diBA4b to D-glucose is a strong evidence of the tighter binding of diBA4b to D-glucose through 2 pairs of diols. FIGS. 15A and B show the results of the titration of diBA4b with D-glucose.

The maximum in UV spectra of diBA4b shifts from 245 to 230 nm. These results show that the synthetic diBA4b has a much higher affinity to D-glucose, compared to its precursor monoBA, and is potentially a better binding moiety for selective and sensitive detection of ³H-D-glucose.

On the other hand, the titration of diBA4b with fructose did not show a significant difference from those of the titration of AFBA with fructose. AFBA and diBA4b have the same affinity to fructose because binding of diBAs to fructose is through the same diols, as with monoBAs. There is no improvement in binding affinity of boronic acids to fructose by scaffolding them into bisdentate binding moieties. FIG. 18 shows the results of the titration of dibA4b with fructose. The maximum in UV spectra of the diBA4b shifts from 245 to 230 nm. Absorbance at 245 nm was followed as a function of the concentration of fructose which shows a dramatic decrease in low mM concentrations of fructose, very similar to the results obtained using AFBA.

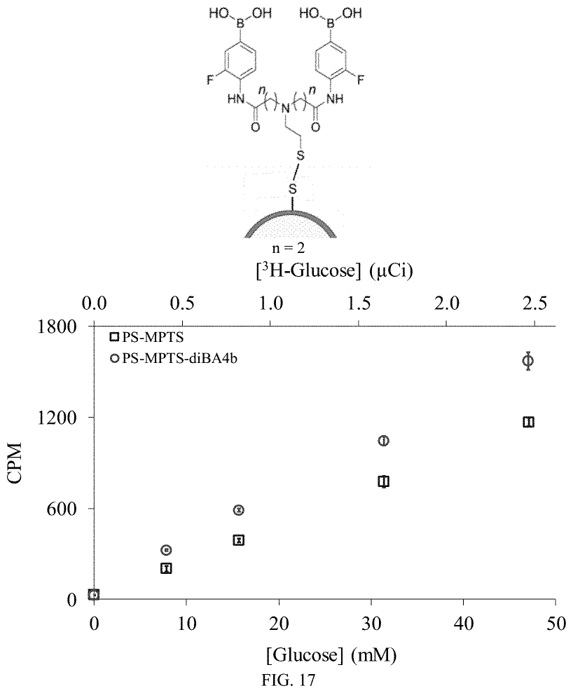

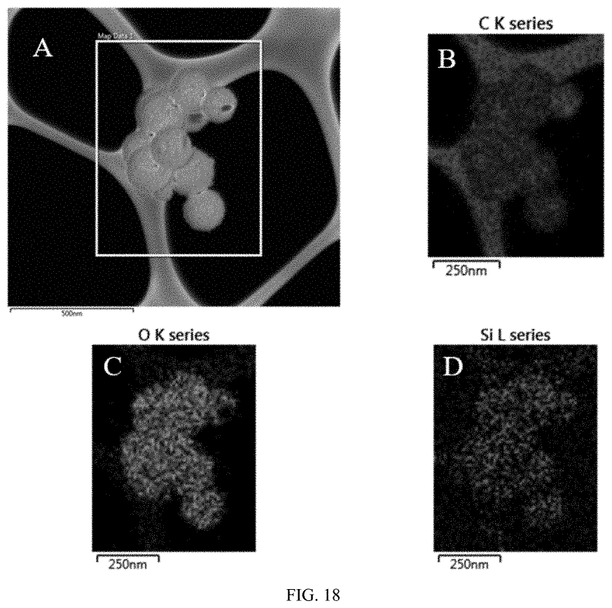

Detection of ³H-D-glucose: DiBA4b was tested as a stronger binding moiety to D-glucose on the surface of scintillant nanoparticles. diBA4b was immobilized on the surface of thiol-functionalized (PS-MPTS) nanoparticles through disulfide binding. The fabricated diBA-nanoSPA was utilized for detection of ³H-D-glucose by titrating a suspension of PS-MPTS-diBA4b nanoparticles with a Glucose-Mix up to 50 mM and 2.5 μCi. A control group of PS-MPTS nanoparticles was included. FIG. 17 shows a schematic structure of diBA-nanoSPA made by immobilizing diBA4b on PS-MPTS nanoparticles and the scintillation responses collected in the SPA analysis.

Scintillation responses of PS-MPTS-diBA4b nanoparticles increased with increasing concentration of D-glucose and they were significantly different from those of the control group (PS-MPTS). Using a Glucose-Mix is the main cause of the poor signal to background ratio. Insufficient immobilization of diBA4b on thiol-functionalized nanoparticles may also be contributing to the insignificant difference of signal from background. Therefore, surface analysis of nanoparticles, using STEM and XPS, was performed to confirm the presence of diBA4b on nanoparticles. STEM imaging of PS-MPTS-diBA4b nanoparticles was performed for elemental analysis. B, F, and N were analyzed, in addition to C, Si, and O, to confirm the immobilization of diBA4b on thiol-functionalized nanoparticles. FIG. 18 shows the results of STEM imaging of diBA-nanoSPA. STEM did not measure detectable levels of B, N, or F on the surface of PS-MPTS-diBA4b nanoparticles. The results of the titration of these nanoparticles with a Glucose-Mix, however, showed a slightly different signal from background. It may be concluded that the diBA4b is immobilized on the surface of PS-MPTS nanoparticles but with insufficient diBA4b binding moieties on the surface which minimizes signal enhancement.

Further investigation of the surface of PS-MPTS-diBA4b nanoparticles was performed using XPS analysis. XPS is a semiquantitative analytical technique that is suitable for characterization of surface composition with higher sensitivity and resolution, compared to STEM imaging. XPS is based on the interaction of a monochromatic source of X-rays with the specimen under ultrahigh vacuum. Core electrons of the elements on the surface of a specimen are ejected upon interaction of the elements with the incident X-ray light and outer shell electrons are collapsed to the created free spaces, which leads to the release of other X-rays, representative of the elemental composition of the surface, that are detected by an electron analyzer.

Elemental analysis of the surface of PS-MPTS-diBA4b nanoparticles was performed using an Al Kα source and showed N and F on the surface. XPS analysis did not measure detectable B on the surface of these nanoparticles, however, which may be due to the low sensitivity of this technique to low-Z elements. B has a small cross-section, compared to that of N and F, and detection through elemental analysis is challenging. It is concluded that there are diBA4b binding moieties immobilized on the surface of PS-MPTS nanoparticles, however, the concentration is insufficient for sensitive detection of ³H-D-glucose. Moreover, the binding affinity might be altered after immobilization.

Table A presents the quantitative analysis of XPS data. Binding energy (BE) and full width at half maximum (FWHM) of the peaks collected for F, C, S, and N are listed. Raw peak area, relative sensitivity factor (RSF), and atomic masses are used to calculate the concentrations of the elements on the surface of nanoSPA. The analysis of XPS data for PS-MPTS-diBA4b nanoSPA shows 1.13% F and 2.01% N on the surface, which may be improved by a better coupling strategy to immobilize a greater number of diBA4b molecules on the surface of nanoparticles. The quantitative ratio of F:N is approximately 2:3.6 which is about the mole ratio of these elements in the structure of diBA4b (i.e. 2:3).

TABLE A
Elemental analysis of the surface of diBA4b-nanoSPA
(PS-MPTS-diBA4b nanoparticles) using XPS.

| | Position | | Raw Area | | | | |
|---|---|---|---|---|---|---|---|
| Peak | BE | FWHM | (CPS · | | Atomic | Atomic | Mass |
| Type | (eV) | (eV) | eV) | RSF | Mass | Conc % | Conc % |
| F 1s | 685.700 | 0.868 | 146.3 | 1.000 | 18.998 | 0.79 | 1.13 |
| C 1s | 283.300 | 1.726 | 4247.0 | 0.278 | 12.011 | 91.59 | 83.02 |
| S 2p | 162.400 | 2.039 | 618.7 | 0.668 | 32.065 | 5.72 | 13.84 |
| N 1s | 398.700 | 0.814 | 154.0 | 0.477 | 14.007 | 1.90 | 2.01 |

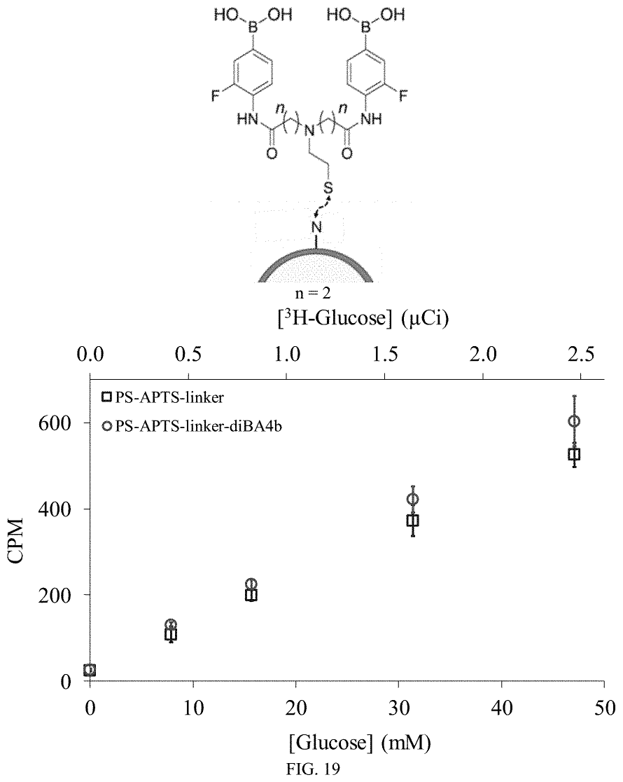

To improve the sensitivity of diBA-nanoSPA to ³H-D-glucose, immobilization of diBA4b on scintillant nanoparticles was repeated through a different coupling chemistry. DiBA4b was immobilized on scintillant amine-functionalized (PS-APTS) nanoSPA using Mal-PEG₂-NHS crosslinker. PS-APTS-diBA4b nanoSPA were utilized for detection of ³H-D-glucose by titrating a suspension of these nanoparticles with a Glucose-Mix up to 50 mM and 2.5 μCi. A control group of PS-APTS nanoSPA with immobilized crosslinkers was included in the SPA experiment. FIG. 19 shows a schematic structure of diBA-nanoSPA made by immobilizing diBA4b on PS-APTS nanoSPA using a crosslinker (double-headed arrow). The scintillation responses of PS-APTS and PS-APTS-diBA4b nanoSPA were collected as a function of the concentration of D-glucose and activity of ³H-D-glucose, as also shown in FIG. 19.

Scintillation responses of PS-APTS-diBA4b nanoSPA increased with increasing concentration of D-glucose, however, they were not significantly different from those of the control group (PS-APTS). As explained before, using a Glucose-Mix is the main cause of the poor signal to background ratio. Nonetheless, the surface concentration of diBA4b on the amine-functionalized nanoparticles was evaluated using STEM-EDX and XPS. STEM-EDX imaging of PS-APTS-diBA4b nanoSPA was performed for elemental analysis of the surface of nanoparticles, as described for PS-MPTS-diBA4b.

N was observed on the surface of the nanoparticles, mostly because of the silica coating of the nanoparticles that contains amine-functionalized silica. However, F was also detected which is a strong evidence of the successful immobilization of diBA4b on amine-functionalized nanoparticles. There was no detectable B on the surface of the amine-functionalized nanoparticles, most probably due to the lower sensitivity of this technique to B, in addition to the low concentration of diBA4b moieties.

Further investigation of the surface of PS-APTS-diBA4b nanoSPA was done by XPS analysis. Elemental analysis of the surface of nanoparticles did not show detectable B, however, a small peak was recorded for F, in addition to a peak significantly higher than background for N. It is concluded that there is some diBA4b immobilized on the surface of nanoparticles, however, the concentration is not high enough. B was not detected due to its lower cross section compared to those of N and F.

Table B presents the quantitative analysis of XPS data obtained for PS-APTS-diBA4b nanoSPA, which shows a slightly higher concentration of F (1.32%) on the surface of PS-APTS-diBA4b nanoSPA, compared to that of PS-MPTS-diBA4b nanoparticles. The significantly higher concentration of N on the surface is due to the amine functional groups on the silica coating of nanoSPA. Therefore, the ratio of F:N may not be attributed to the ratio of the elements in diBA4b moiety. It may be concluded that using the crosslinker slightly improved the affiliation of diBA4b molecules to the surface of scintillant PS-APTS-diBA4b nanoSPA, as the disulfide binding in PS-MPTS-diBA4b nanoparticles is less stable and more susceptible to break which leads to displacement of diBA4b molecules from the surface of nanoparticles.

TABLE B
Elemental analysis of the surface of diBA-
nanoSPA (PS-APTS-DiBA4b nanoSPA) using XPS.

| | Position | | Raw Area | | | | |
|---|---|---|---|---|---|---|---|
| Peak | BE | FWHM | (CPS · | | Atomic | Atomic | Mass |
| Type | (eV) | (eV) | eV) | RSF | Mass | Conc % | Conc % |
| F 1s | 688.036 | 1.092 | 136.9 | 1.000 | 18.998 | 0.85 | 1.32 |
| C 1s | 285.036 | 1.922 | 3529.0 | 0.278 | 12.011 | 88.43 | 86.46 |
| N 1s | 400.436 | 2.149 | 748.0 | 0.477 | 14.007 | 10.72 | 12.22 |

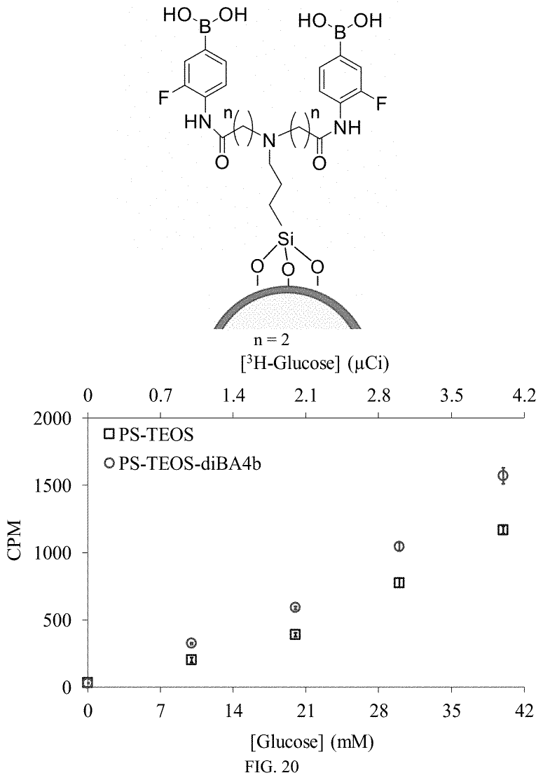

To improve the surface coverage of scintillant core-shell nanoparticles with diBA moieties, a diBA derivative was synthesized using AFBA precursors, but with a silane coupling group, in place of a thiol. The modified diBA is referred to as diBA4bSi and was used in the silica coating to functionalize the surface of the scintillant nanoparticles. PS core nanoparticles were coated with TEOS and newly synthesized diBA4bSi that contained a tri-ethoxy silane group. PS-TEOS-diBA4bSi nanoparticles were prepared and utilized for detection of ³H-D-glucose by titrating a suspension of the nanoparticles with a Glucose-Mix up to 50 mM and 2.5 μCi. A control group of PS-TEOS nanoparticles was included in the SPA experiment. FIG. 20 shows a schematic structure of PS-TEOS-diBA4bSi nanoparticles, made by immobilizing diBA4bSi on PS-TEOS nanoparticles, through silica coating, and the scintillation responses collected in the SPA analysis. Scintillation responses of PS-TEOS-diBA4bSi nanoparticles increased with increasing concentration of D-glucose and were significantly different from those of the control group (PS-TEOS). However, similar to the previous sensors reported based on diBA4b (i.e. PS-MPTS-diBA4b and PS-APTS-diBA4b nanoSPA) the signal enhancement was poor. Using a Glucose-Mix is the main cause of the poor signal to background ratio in addition to unsuccessful or insufficient immobilization of diBA4bSi molecules on nanoparticles.

Conclusions: Fabrication, characterization, and utilization of boronic acid-functionalized nanoSPA platforms were described. Detection of ³H-D-glucose was investigated using nanoSPA functionalized with several monoBAs and diBAs. The summary of data analysis of monoBA- and diBA-nanoSPA platforms are tabulated in Table C. The signal enhancement from the specific binding of nanoSPA to ³H-D-glucose was up to 2.2-fold. The poor signal to background ratio observed in the D-glucose assays is mostly due to the extremely low concentrations of ³H-D-glucose (less than 100 nM).

TABLE C
Data analysis of monoBA- and diBA-nanoSPA platforms.
SPA and NPE counts are reported at 1 μCi ³H-glucose.
LODs are reported based on SPA responses.

| | SPA | NPE | Signal/ | LOD | LOD |
|---|---|---|---|---|---|
| nanoSPA platform | (CPM) | (CPM) | Background | (nCi) | (mM) |
| PS-APTS10%-CBBA | 68 | 47 | 1.5 | 170 | 1.5 |
| PS-APTS25%-CBBA | 260 | 157 | 1.7 | 20 | 1.2 |
| PS-APTS25%-CBBA | 330 | 150 | 2.2 | 25 | 0.1 |
| PS-APTS50%-CBBA | 334 | 341 | 1.0 | 23 | 0.2 |
| PS-MPTS-VPBA | 591 | 421 | 1.4 | 30 | 0.3 |
| PS-MPTS-APBA | 127 | 112 | 1.1 | 48 | 1.0 |
| PS-APTS-diBA4b | 265 | 232 | 1.1 | 79 | 1.5 |
| PS-MPTS-diBA4b | 669 | 481 | 1.4 | 12 | 0.2 |
| PS-TEOS-diBA4bSi | 330 | 227 | 1.5 | 19 | 0.2 |

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

1-30. (canceled)

31. A silica shell encapsulated composite material adapted for use in detection of a saccharide, said silica shell encapsulated composite material comprising:
an organic polymer core portion (1);
a silica shell portion (2) encapsulating said organic polymer core portion (1);
a polymer (3) attached to the surface of said silica shell portion (2); and
a boronic acid functional group (6) attached to the surface of said silica shell portion (2).

32. The silica shell encapsulated composite material of claim 31, wherein said boronic acid functional group (6) is attached to the surface of said silica shell portion via a linker (5).

33. The silica shell encapsulated composite material of claim 32, wherein said linker (5) comprises a plurality of said boronic acid functional groups (6).

34. The silica shell encapsulated composite material of claim 31, wherein said composite material is a microcomposite material.

35. The silica shell encapsulated composite material of claim 31, wherein said composite material is a nanocomposite material.

36. The silica shell encapsulated composite material of claim 31, wherein said organic polymer core portion (1) further comprises at least one scintillator material.

37. The silica shell encapsulated composite material of claim 36, wherein said scintillator material comprises p-terphenyl (PTP); 1,4-bis (4-methyl-5-phenyl oxazolyl)benzene (dimethyl POPOP), or a mixture thereof.

38. The silica shell encapsulated composite material of claim 31, wherein said organic polymer core portion (1) comprises polystyrene, polyvinyltoluene (PVT), polyphenylethers (PPE), polyvinylcarbazole (PVK), or a combination or mixture thereof.

39. The silica shell encapsulated composite material of claim 31, wherein said organic polymer core portion (1) has mean particle size of about 300 nm or less.

40. The silica shell encapsulated composite material of claim 31, wherein the average particle size of said organic polymer core portion (1) ranges from about 20 nm to about 1.5 μm.

41. The silica shell encapsulated composite material of claim 31, wherein the thickness of said silica shell portion (2) ranges from about 10 nm to about 250 nm.

42. A method for detecting a level of a saccharide in a sample, said method comprising:
contacting said sample with a silica shell encapsulated composite material of claim 1 in the presence of a signal generator under conditions sufficient to produce a signal when said saccharide is present in said sample; and
analyzing the signal to determine the level of saccharide present in said sample.

43. The method of claim 42, wherein said sample comprises blood, serum, saliva, urine, a cell, a cell lysate, a microorgan, or a tissue.

44. The method of claim 42, wherein said step of analyzing the signal comprises determining the amount of fluorescence, UV-Vis absorption, infrared absorption, phosphorescence, Raman spectroscopy, radioactivity scintillation, radioisotope decay or electrochemistry.

45. The method of claim 42, wherein said signal generator comprises a radioactive material.

46. The method of claim 42, wherein said organic polymer core portion comprises a scintillator material.

47. The method of claim claim 46, wherein said method comprises a scintillation proximity assay.

48. The method of claim 46, wherein said silica shell encapsulated composite material comprises said boronic acid functional group (6) that is attached to the surface of said silica shell portion (2) via a linker (5).

49. A method for determining a glucose level in a sample obtained from a subject, said method comprising the steps of:
contacting said sample with a silica shell encapsulated composite material comprising:
an organic polymer core portion (1), wherein said organic polymer core portion comprises at least one scintillator material;
a silica shell portion (2) encapsulating said organic polymer core portion (1);
a polymer (3) attached to the surface of said silica shell portion (2); and
a boronic acid functional group (6) attached to the surface of said silica shell portion (2), wherein said boronic acid functional group (6) binds to glucose when glucose is present in said sample;
determining the glucose level in said sample by analyzing the amount of glucose binding to said boronic acid functional group.

50. The method of claim 49, wherein said step of determining the glucose level is comprises a scintillation proximity assay.
